(19) United States
(12) Patent Application Publication
(10) Pub. No.: US 2006/0240430 A1
(43) Pub. Date: Oct. 26, 2006

(54) Method for hybridisation of immobilized genomic dna

(76) Inventor: Ying Wu, Nijmegan (NL)

Correspondence Address: AMSTER, ROTHSTEIN & EBENSTEIN LLP, 90 PARK AVENUE, NEW YORK, NY, 10016, US

(21) Appl. No.: 10/537,149

(22) PCT Filed: Dec. 2, 2003
(86) PCT No.: PCT/EP03/13601

§ 371 Date: Dec. 22, 2005

Related U.S. Application Data
(60) Provisional application No. 60/440,689, filed on Jan. 17, 2003.

(30) Foreign Application Priority Data
Dec. 4, 2002 (EP) 02447241.7

(51) Int. Cl. C12Q 1/68; C12M 1/34

(52) U.S. Cl. 435/6; 435/287.2

(57) ABSTRACT

The present invention is directed to a novel method of efficiently hybridising probes onto immobilized genomic DNA and/or RNA comprising the steps of (a) providing intact genomic DNA and denaturing said intact genomic DNA; (b) immobilizing said denatured intact genomic DNA onto a matrix; said matrix comprising pore sizes within a range of 0.6 μm to 2 μm including the outer limits (c) providing a set of probes and passing said probes through said matrix under conditions favouring hybridisation of the probes to its complementary sequence in said intact genomic DNA; and (d) washing off non-hybridised probes through said matrix, leaving formed hybridised intact genomic DNA/probe complexes for further analysis. The present invention is further directed to a novel method for target nucleic acid detection and quantification in a genomic DNA sample comprising the steps of: (a) providing intact genomic DNA and denaturing said intact genomic DNA; (b) performing a hybridisation according to a method as described above; (c) recovering hybridised probes; and essentially simultaneously amplifying any recovered probe using a single primer pair, each member of said primer pair binding to each recovered probe onto the respective flanking primer attachment sequences of said probe, and (d) qualitatively and quantitatively analysing the recovered amplified probes of step (c). The present invention also relates to the uses thereof as well as devices, apparatus and kits for performing said methods of the invention.

### FIELD OF THE INVENTION

The present invention relates to methods for intact genomic nucleic acid material hybridisations and detection and quantification of target nucleic acids in a genomic DNA sample.

The present invention relates in particular to methods for automated multiple amplifiable probe hybridisations onto genomic DNA.

The methods of the present invention are particularly useful in screening methods for detection of copy number and changes in copy number of genomic DNA.

### BACKGROUND OF THE INVENTION

Abnormalities of DNA copy number account for many genetic diseases in living organisms, including many human genetic disorders. The largest of these abnormalities involve changes in copy number in entire chromosomes; for example in monosomies and trisomies (for example trisomy 21 resulting in Down syndrome), and segmental abnormalities such as 5 p deletion in cri-du-chat syndrome. Alternatively, genetic diseases such as for example DMD (Duchenne muscular dystrophy), BRCA1 (breast cancer) or MSH2/MLH1 (hereditary nonpolyposis colorectal cancer or HNPCC) may evolve from smaller copy number changes in genomic DNA which are too small to be detected by conventional cytogenetics. Further, at the level of individual genes, specific inherited diseases can result from deletions or duplications involving individual exons or entire genes.

The detection of changes in copy number in a complex genome is not straightforward. Commonly applied methods for diagnosing genetic diseases due to copy number alterations involve for example quantitative multiple PCR, Southern blotting and comparative genomic hybridisation. These techniques, however, albeit commonly practiced and to a great extend recognized for their reliability are subject to a number of disadvantages. Southern blotting is time consuming and duplications may be difficult to be detected. A disadvantage of multiplex PCR for example is its restriction to the number of loci which can be analysed simultaneously. Although comparative genomic hybridisation can analyse a whole genome in a single test, resolutions were proven to be relatively low. It is clear from the above that detection of copy number in a complex genome has a great technical challenge.

Despite its fundamental importance, it is only recently that systematic approaches have been developed to assess copy number at specific genetic loci, or to examine intact genomic DNA for sub-microscopic deletions of unknown location. New approaches include for example multiplex amplifiable probe hybridisation as described in WO 00/53804 (Armour). Although the power and specificity of multiplex amplifiable probe hybridisation is proven by the simultaneous assessment of copy number at large sets of human loci, this technique suffers from the general disadvantage that in particular the handling step with regard to removal of unbound probes is quite time-consuming.

Due to the exponential growth of research activity and diagnostic development, demand for improved hybridisation procedures is imperative and those skilled in the art recognize that it would be a distinct advantage in diagnostic research and extremely beneficial in commercial diagnostics if a highly efficient and economic diagnostic tool would be available.

Although flow-through hybridisation methods known in the art have become appreciated over the last years for their efficiency in performing numerous analysis technologies, such methods are restricted to the hybridisation of probes to four types of nucleic acid probes: large sections of DNA, small DNA (including cDNA), RNA, and peptide nucleic acids.

The present invention describes the principle of a unique flow-through hybridisation process for immobilized undigested or intact genomic DNA and a device for the said purpose whereby the hybridisation time as well as the amount of reagents used for hybridisation can be reduced by many folds. In particular, the present invention enables analysis of undigested or intact genomic DNA, thus not requiring time-consuming pre-hybridisation manipulation steps such as required in fragmentation-based procedures.

The present invention aims at providing improved methods for the quantitative detection of nucleic acids in a genomic sample with high resolution.

It is a further object of the present invention to provide methods for the quantitative detection of nucleic acids in a genomic sample with a much improved sensitivity level.

It is a further object of the present invention to provide methods for the quantitative detection of nucleic acids in a sample possessing an improved time-management.

It is a further object to provide devices, apparatuses and kits for carrying out said methods.

### SUMMARY OF THE INVENTION

In order to accurately quantify nucleic acids within a genomic nucleic acids sample, the present invention provides a method for hybridisation of probes onto immobilized intact genomic DNA comprising the steps of (a) providing intact genomic DNA and denaturing said intact genomic DNA; (b) immobilizing said denatured intact genomic DNA onto a matrix, said matrix comprising pore sizes within a range of 0.6 μm to 2 μm including the outer limits; (c) providing a set of probes and passing said probes through said matrix under conditions favouring hybridisation of the probes to its complementary sequence in said intact genomic DNA; and (d) washing off non-hybridised probes through said matrix, leaving formed hybridised intact genomic DNA/probe complexes for further analysis.

The present invention provides methods for flow-through genomic hybridisation which are fast (high-speed), highly sensitive, highly specific and miniaturized.

The present invention allows much decreased analysis time by using flow-through hybridisation technology combined with the use of undigested or undigested or intact or non-manipulated genomic DNA. As exemplified within the present specification, non-routine experimentation led to the surprising finding that only matrices with specific parameters fulfil the requirements of passing said probes through said matrix to its complementary sequence in said intact genomic DNA while assuring the most favourable hybridisation kinetics.

A general outline of the hybridisation methods provided by the present invention is given in FIG. 1.

### DETAILED DESCRIPTION OF THE INVENTION

Before the present method and solutions used in the method are described, it is to be understood that this invention is not limited to particular methods, components, or solutions described, as such methods, components, and solutions may, of course, vary.

In the present specification and the appended claims, the singular forms “a”, “an”, and “the” include plural references unless the context clearly dictates otherwise.

It should also be understood that the terminology used herein is not intended to be limiting, since the scope of the present invention will be limited only by the appended claims.

Accordingly, definitions should not be understood to limit the scope of the invention. Rather, they should be used to interpret the language of the description and, where appropriate, the language of the claims. These terms may also be understood more fully in the context of the description of the invention. If a term is included in the description or the claims that is not further defined within the present description, or that cannot be interpreted based on its context, then it should be construed to have the same meaning as it is understood by those skilled in the art.

The present invention relates in particular to screening for sequence copy number and to screening for changes in copy number of a plurality of nucleic acid sequences in a genome or genomic sample.

The terms “genome”, “genomic content”, “genomic sample”, “genomic DNA”, “genomic nucleic acid material” and “genetic material” are used interchangeably throughout the present specification and mean the nucleic acid molecules in an organism or cell that are the ultimate source of heritable genetic information of the organism. For most organisms, a genome consists primarily of chromosomal DNA, but it can also include plasmids, mitochondrial DNA, and so on. For some organisms, such as RNA viruses, a genome consists of RNA. As used within the present specification, genomic DNA is undigested or intact unless otherwise stated.

The terms ‘undigested genomic DNA’ and ‘intact genomic DNA’ are used interchangeably throughout the present specification.

By “nucleic acid” is meant DNA, RNA, or other related compositions of matter that may include substitution of similar moieties. For example, nucleic acids may include bases that are not found in DNA or RNA, including, but not limited to, xanthine, inosine, uracil in DNA, thymine in RNA, hypoxanthine, and so on. Nucleic acids may also include chemical modifications of phosphate or sugar moieties, which can be introduced to improve stability, resistance to enzymatic degradation, or some other useful property.

The loss or reduction in the normal number of copies of a genetic sequence (deletion) or the increase in copy number (amplification) are of widespread general importance. Such genetic alterations are known to underlie phenotype characteristics both somatic and germline. The demonstration of the site and nature of such genetic alteration is critical in the identification of the genes responsible and to the development of appropriate and effective treatments and therapies.

The present invention provides for methods to obtain genetic information from samples containing or suspected to have genomic content. It is medically and/or environmentally and/or socially important to identify genomic disorders. It will be well appreciated that also e.g. infectious organisms may be identified and quantified in such samples for optimal treatment of infections or contamination and for maintaining public health.

Methods according to the present invention are particularly designed for probe hybridisation onto immobilized genomic nucleic acid material. Hybridisation methods according to the present invention are characterized in that undigested or intact genomic contents are immobilized and subjected to flow-through probe hybridisation techniques.

Within this context, it is another object of the invention to provide for the use of a method according to the present specification and as described herein for intact genomic DNA hybridisation.

The immobilized genetic material within the present invention originates from a sample to be analysed for the presence/absence of any genomic abnormality.

By the term “genomic abnormality” is meant any deviation from a normal genomic content status. A genomic content status characterizes the condition or part thereof of a sample or the corresponding whole from which said genomic content was identified and quantified. A genomic abnormality may prevail through for example mutation(s) at the level of entire chromosomes including deletions and duplications, segmental abnormalities, genomic DNA deletions and duplications at the level of individual genes, involving individual exons or entire genes.

Abnormalities or irregularities involving endogenes as well as exogenes may lead to genomic abnormalities.

Accordingly, a genomic abnormality may equally prevail through the presence of exogenous nucleic acids such as by way of example and not limitation: naked autonomous replicating nucleic acids including for example plasmids and viroids; and embodied autonomous replicating nucleic acids such as pathogens, parasites, and contaminants. Said pathogen, parasite, and contaminant may be algae, archaea, bacteria; viruses; fungi including yeasts, molds and mycorrhizae; nematodes; protozoa and microsporidae.

A sample containing or suspected to have a genomic content may be biological material or any material comprising biological material from which nucleic acids may be prepared and analysed for the qualitative and quantitative presence of particular nucleic acid sequences. Genomic nucleic acid material to be used in the methods of the present invention may be within its sample format for direct analysis. However, a particular useful format is provided when the sample is subjected to some preparation prior to use in the analysis of the present invention. Said preparation may involve the removal of non-nucleic acid debris and suspension/dilution of the pure or isolated nucleic acid material in water or an appropriate buffer.

The genomic material may be isolated from virtually any sample. However, usually, the sample is a biological or a biochemical sample. The term “biological sample,” as used herein, refers to a sample obtained from an organism or from components (e.g., cells) of an organism. The sample may be of any biological tissue or fluid. Frequently the sample will be a “clinical sample” which is a sample derived from a patient. Such samples include, but are not limited to, sputum, cerebrospinal fluid, blood, blood fractions such as serum including foetal serum (e.g., SFC) and plasma, blood cells (e.g., white cells), tissue or fine needle biopsy samples, urine, peritoneal fluid, and pleural fluid, or cells there from. Biological samples may also include sections of tissues.

Hybridisation methods according to the present invention use intact genomic DNA that is isolated. Methods for genomic DNA isolation from various samples are well known in the art.

Typically, intact genomic DNA is denatured prior to immobilization. DNA may be denatured by boiling or other methods as well known in the art.

The denatured DNA is subsequently immobilized within a matrix.

The term “matrix” refers to a material in which genetic material may be enclosed or embedded (as for study). The term matrix encompasses a wide range of potential substrates that can be used for the immobilization of intact genomic DNA.

Generally, the matrix can be composed of any material which will permit immobilization of intact genomic DNA or nucleic acids and which will not melt or otherwise substantially degrade under the conditions used to immobilize said genomic material and which allows hybridisation of said immobilized genomic material with probes by flow-through hybridisation.

A number of materials suitable for use as matrices in the present invention have been described in the art. Materials particularly suitable for use as matrices in the present invention include any type of permeable synthetic materials or natural materials provided that the pore diameter in case of a porous matrix or the mesh size in case of a matrix network allow for the permeation of the intact genomic nucleic acid material. Suitable matrix materials have pore sizes comprised within a range of 0.6 μm to 2.0 μm including the outer limits; e.g. 0.6, 0.7, 0.8, 0.9, 1.0, 1.1, 1.2, 1.3, 1.4, 1.5, 1.6, 1.7, 1.8, 1.9, and 2.0 μm. Particular suitable pores sizes are comprised within a range of 0.6 μm to 1.2 μm including the outer limits; e.g. 0.6, 0.7, 0.8, 0.9, 1.0, 1.1 and 1.2 μm. One of suitable materials as exemplified within the present specification is Whatman 3MM Chr paper. This exemplified material should however not be taken as in any way limiting. It will be well appreciated by a person skilled in the art that matrix thickness may vary in function of matrix strength, i.e. the thinner a matrix may be; the stronger the material is, wherein thin material might still support flow-through hybridisation and/or flow-through of reaction components. A particular suitable matrix is a thin pore matrix. Advantages, among others, of such particular suitable matrix is the limited amount of genomic DNA material needed. A particular suitable matrix thickness is within a range of 0.1 mm to 1 mm, including the outer limits. A more particular suitable matrix thickness is within a range of 0.3 mm to 0.5 mm including the outer limits.

It will be well appreciated that a combination of matrix materials may be envisaged in obtaining a desired matrix format characterized by a desired pore size(s) and strength.

Accordingly, in one embodiment of the present invention, a hybridisation method is provided, wherein said denatured intact genomic DNA is permeated within said matrix.

A matrix may be in the form of sheets, films or membranes and are permeable. For example, a matrix may consist of fibres such as glass wool or other glass or synthetic fibres such as plastic fibres, polyamide fibres (e.g. nylon) and the like. A matrix may equally consist of animal fibres such as silk and wool, or plant or vegetable fibres such as cotton, cellulose fibres and nitrocellulose fibres or cellulosic fibres including for example acetate and triacetate.

The matrix may be planar or have a simple or a complex shape. Particular useful matrices are membranes comprising a 3D network structure of which the surface to which the genomic nucleic acids are adhered is external surface as well as internal surface of the matrix. However, as will be appreciated in the art, it will be predominantly the internal surfaces that will have adhered thereto the genomic DNA.

Accordingly, in one embodiment of the present invention, a hybridisation method is provided, wherein the matrix is a membrane.

In a further embodiment of the present invention, a hybridisation method is provided, wherein said membrane comprises a 3D network structure.

In yet a further embodiment of the present invention, a hybridisation method is provided, wherein said network structure is a fibre network structure.

In yet a further embodiment of the present invention, a hybridisation method is provided, wherein said fibre is of vegetable origin.

In yet a further embodiment of the present invention, a hybridisation method provided, wherein said fibre is cellulose.

The principle of the present invention is using a flow-through mechanism by which the probes pass through the membrane structure, allowing these probes coming in close contact with the corresponding complementary sequences within the immobilized genomic nucleic acids so that the target sequences can be effectively detected in high sensitivity and specificity.

Accordingly, in one embodiment of the present invention, a hybridisation method is provided, wherein said network structure is a flow-through structure.

Whatever the substrate or matrix material, there are a number of ways that nucleic acids can be attached or immobilized. Most common are physical adsorptive processes or chemical linking processes, including ultraviolet (UV) or covalent methods.

A great deal of interest has been shown in the production of activated membranes, that is, membranes that have direct reaction chemistry available on their surfaces. The ability to apply an aqueous sample directly to the membrane, and to have binding occur naturally without the need for further reagents, offers significant advantages, these are especially known with respect to the production of microarrays. Several different activation chemistries are well known in the art.

Accordingly, in one embodiment of the present invention, a hybridisation method is provided, wherein the matrix is activated with an affinity conjugate.

In a further embodiment, a hybridisation method is provided, wherein said affinity conjugate is chosen from the group comprising poly-L-lysine, poly-D-lysine, 3-aminopropyl-triethoxysilane, poly-arginine, polyethyleneimine, polyvinylamine, polyallylamine, tetraethylenepentamine, ethylenediamine, diethylenetriamine, triethylenetetramine, pentaethylenehexamine and hexamethylenediamine.

In yet a further embodiment, a hybridisation method is provided, wherein said affinity conjugate is poly-L-lysine.

Handling of the matrix is greatly improved by means of a device for holding said matrix such as described in PCT/EP02/02446 which is herewith incorporated by reference.

High-throughput analysis of numerous samples simultaneously may be accommodated by a system such as described in European Application No. 02076728.1, herewith incorporated by reference, which discloses a system for conducting bioassays, comprising a substrate plate with a number of wells, and an incubation device for holding the plate. This known analytical test device is composed of a plastic support wherein openings in the plastic support define wells with a certain diameter, said wells being open at the top for sample or probe application and having a substrate defining the bottom of each well. Said substrate may be a matrix as described in the present specification.

A system as described above allows for parallel processing of a large number of genomic nucleic acid samples and may be applied in automated robotic platforms. Such system usually comprises a microplate with an array of wells arranged in rows and columns, wherein the bottom of each well is a matrix having a flow-through fibre network. Using for example a microplate with an array of ninety-six wells allows a parallel processing of a large number of hybridisations resulting in a very efficient high-throughput analysis.

Accordingly, it is an object of the present invention to provide a device for flow-through hybridisation of probes onto immobilized intact genomic DNA comprising a well holder, said well holder comprising one or more round wells with a fixed diameter, said wells exposing a fibre network matrix, said matrix comprising pore sizes within a range of 0.6 μm to 2 μm including the outer limits; wherein said matrix permits immobilization of intact genomic DNA and which allows hybridisation of said immobilized intact genomic material with probes by flow-through hybridisation.

In one embodiment of the present invention, a device for flow-through hybridisation of probes onto immobilized genomic DNA is provided wherein said matrix permits permeation of intact genomic DNA.

Application of a pressure difference over the matrix will force the probes through the matrix 3D network structure, thereby creating a low pressure within the wells. By removing said low pressure, the probes are automatically forced back through the network of the matrix. Higher pressures will create a more rapid flow of the probes through the matrix structure. By alternatingly creating a low pressure over the matrix and removing the low pressure, the probes are forced through the matrix network a number of times.

Accordingly, it is another object of the present invention to provide an apparatus for flow-through hybridisation of probes onto immobilized intact genomic DNA comprising:
(a) a device for flow-through hybridisation of probes onto immobilized intact genomic DNA comprising a well holder, said well holder comprising one or more round wells with a fixed diameter, said wells exposing a fibre network matrix;
(b) means for addition of a controlled amount of fluid to at least one of the wells of the device as described in (a);
(c) means for applying and/or maintaining a controlled pressure difference over the matrix in each of the wells.

An optimum flow-rate is such that the residence time of the probe molecules near the immobilized target sequences is sufficient to generate hybridisation events in the shortest possible time.

Hybridisations are usually performed with flow rates comprised between 50 mm/30 min and 250 mm/30 min including the outer limits. Particular suitable flow rates are comprised between 75 mm/30 min and 200 mm/30 min including the outer limits. More particular suitable flow rates are comprised between 100 mm/30 min and 150 mm/30 min including the outer limits. Usually, a particular suitable flow rate is 130 mm/30 min including the outer limits.

Accordingly, in one embodiment of the present invention, hybridisation methods are provided, wherein the matrix allows for a flow rate comprised between 50 mm/30 min and 250 mm/30 min including the outer limits.

A flow-through incubation, as employed in the methods as described herein, gives significantly reduced hybridisation times. Positive or negative pressure may be applied to the matrix in order to pump the probe solution dynamically up and down through the matrix pores or matrix network which may enhance the diffusion of the probes to the target sequences within the immobilized genomic material.

The duration of one cycle of forward and backward flow of probe solution across the matrix membrane may be comprised between 10 min and 1 sec. Usually, duration of one cycle is comprised between 5 min and 10 sec. More usually, duration of one cycle is comprised between 5 min and 10 sec. Yet more usually, duration of one cycle is comprised between 1 min and 45 sec. A particular suitable example of duration of a single cycle of forward and backward flow is 30 sec.

In another embodiment of the present invention, a hybridisation method is provided, wherein said probes are passed through said matrix by at least one cycle of alternating downwards and upwards flow.

It is common to perform analysis at a single constant temperature; the preferred temperature will depend on the envisaged hybridisation stringency. Adjustment of the hybridisation temperature may be accomplished by coupling of the matrix via a holding device to a heating device such as a water bath or a conductive heating plate. Alternatively, an incubator system with a temperature control system may be provided wherein said holding device comprising one or more matrices may be housed. Such incubator system is described in for example PCT/EP02/02448 which is hereby incorporated by reference.

Once sufficient time has elapsed or sufficient flow cycles have elapsed to provide for hybridisation, unbound probes are washed thoroughly away by means of a post-hybridisation flow-through wash step. A dynamical pumping allows immediate and highly efficient removal of any unbound probe in as little as one single downward flow. In general, flow conditions with regard to number of cycles and flow speed may be varied according to the envisaged stringency.

In one embodiment of the present invention, a hybridisation method is provided, wherein the washing step is carried out by passing through the matrix a wash fluid by at least one cycle of downwards flow.

Any bound probe is subsequently recovered and amplified. Said recovering and amplification may be essentially simultaneously, i.e. the probe-recovering step may be performed in a nucleic acid amplification environment. For example, nucleic acids immobilized onto a membrane and bound to identifier probes may be immersed in nucleic acid amplification buffer comprising amplification components. Setting of denaturing conditions will set free the bound identifier probes which then may be essentially simultaneously amplified.

Probe amplification involves the amplification (i.e. replication) of the identifier probe sequence being bound to the immobilized sample genomic nucleic acids, resulting in a significant increase in the number of identifier probe molecules.

Although numerous amplification techniques are known in the art, a particular suitable amplification technique employs a single primer pair, whereby each member of said primer pair is complementary to a primer binding sequence which is positioned flanking 5′ or 3′ to each identifier probe; said 5′ and 3′ flanking primer binding sequences being the same or substantially the same for each probe.

Accordingly, in one embodiment of the present invention, a method for hybridisation of probes onto immobilized intact genomic DNA is provided wherein the probes are flanked by primer binding sequences,

As used herein, “amplification” refers to the increase in the number of copies of a particular nucleic acid of interest wherein said copies are also called “amplicons” or “amplification products”. In particular, by amplification is meant a technique for linearly or exponentially increasing the copy number of a nucleic acid molecule.

In one embodiment of the present invention, a hybridisation method is provided wherein the amplification of the recovered hybridised probes is a quantitative amplification.

The requirement for sensitivity (i.e. low detection limits) has been greatly alleviated by the development of the polymerase chain reaction (PCR) and other amplification technologies, which allow researchers to amplify exponentially a specific nucleic acid sequence before analysis. Suitable amplification methods include exponential amplification methods such as for example PCR, quantitative PCR (Q-PCR), biotin capture PCR as well as linear amplification methods such as for example linear amplification by in vitro transcription TYRAS and NASBA.

In a further embodiment of the present invention, a hybridisation method is provided wherein said amplification is by means of polymerase chain reaction.

The polymerase chain reaction (PCR) is widely used and described, and involves the use of primer extension combined with thermal cycling to amplify a target sequence; see U.S. Pat. No. 4,683,195 and U.S. Pat. No. 4,683,202, and PCR Essential Data, J. W. Wiley & sons, Ed. C. R. Newton, 1995, all of which are incorporated by reference.

Suitable PCR-based amplification strategies are well known in the art and may be, by way of example and not limitation, routine quantitative PCR (QC-PCR), reverse transcriptase PCR or RT-PCR, biotin capture PCR, nucleic acid sequence based amplification (NASBA), and TYRAS.

The TYRAS amplification method as disclosed in WO 99/43850, hereby incorporated by reference, is a non-selective poly-A mRNA amplification method which does not encompass cDNA synthesis. The method comprises the hybridisation of an oligonucleotide, encompassing an oligo-T stretch, to the poly-A tail of the mRNA followed by RNase H digestion opposite the oligonucleotide and extension of the newly formed 3′ end of the mRNA with reverse transcriptase. In this way the T7 RNA polymerase recognition sequence (i.e. T7 promoter) that is part of the oligonucleotide encompassing an oligo-T stretch is made double stranded. Upon binding of the T7 RNA polymerase to the promoter the original mRNA molecules are transcribed in multiple RNA copies of the opposite polarity.

RNA may also be amplified according to the method as disclosed in U.S. Pat. No. 5,545,522 (Van Gelder), hereby incorporated by reference, wherein cDNA is synthesized from an RNA sequence using a complementary primer linked to an RNA polymerase promoter region complement and then antisense RNA (aRNA) is transcribed from the cDNA by introducing an RNA polymerase capable of binding to the promoter region.

Nucleic acid sequence based amplification (NASBA) is generally described in U.S. Pat. No. 5,409,818 and “Profiting from Gene-based Diagnostics”, CTB International Publishing Inc., N.J., 1996, both of which are incorporated by reference. NASBA relies on the simultaneous activity of 3 enzymes: AMV-RT (Avian Myoblastosis Virus-Reverse Transcriptase), RNase H and T7 RNA polymerase. NASBA is a special case of the 3SR amplification reaction or self-sustained sequence replication-reaction.

The 3SR reaction is a very efficient method for isothermal amplification of target DNA or RNA sequences in vitro. This method requires three enzymatic activities: reverse transcriptase, DNA-dependent RNA polymerase and Escherichia coli ribonuclease H.

For use in multiplex PCR, a primer should be designed so that its predicted hybridisation kinetics are similar to those of the other primers used in the same multiplex reaction. While the annealing temperatures and primer concentrations may be calculated to some degree, conditions generally have to be empirically determined for each multiplex reaction. Since the possibility of non-specific priming increases with each additional primer pair, conditions must be modified as necessary as individual primer sets are added. Moreover, artefacts that result from competition for resources (e.g. depletion of primers) are augmented in multiplex PCR, since differences in the yields of unequally amplified fragments are enhanced with each cycle.

As well known by the person skilled in the art, probe design for multiplex PCR may encompass flanking primer binding sequences that do not hybridise to the target sequence to overcome the above-mentioned draw-backs.

Accordingly, in one embodiment of the present invention, a method for target nucleic acid detection and quantification in an intact genomic DNA sample is provided, wherein each probe is flanked 5′ and 3′ by primer binding regions with said 5′ and 3′ flanking primer binding sequences being the same or substantially the same for each probe.

By target sequence or target nucleic acid is meant a nucleic acid sequence that a probe is designed to detect; e.g., for an “identification”-probe, the target sequence might be an identification sequence. By identification sequence is meant a nucleic acid sequence that is diagnostic of a particular organism or group of organisms or that is diagnostic for a particular genetic disease state when its presence or existence is assayed in a genome or enriched genome by hybridisation using the appropriate melting temperature criteria. By an enriched genome or enriched genomic fraction, is meant a genome or genomic fraction that has undergone an enrichment procedure that generates a selected fraction of the original genome. For example, for the purpose of genomic profiling, enriched genomes offer robust hybridisation-based diagnostics.

The set of identifier or identification probes or polynucleotides may correspond to particular mutations that are to be identified in a known sequence. As such, for a known gene that may contain any of several possible identified mutations, the set can comprise polynucleotides corresponding to the different possible mutations. This is, for instance, useful for genes like oncogenes and tumour suppressors, which frequently have a variety of known mutations in different positions.

In one embodiment of the present invention, a method for target nucleic acid detection and quantification in an intact genomic DNA sample is provided, wherein the amplified probes are provided with a label.

The term label as used in the present specification refers to a molecule propagating a signal to aid in detection and quantification. Said signal may be detected either visually (e.g., because it has a coloured product, or emits fluorescence) or by use of a detector that detects properties of the reporter molecule (e.g., radioactivity, magnetic field, etc.). In the present specification, labels allow for the detection of the identification and quantification of target sequences within an intact genomic sample. Detectable labels suitable for use in the present invention include but are not limited to any composition detectable by spectroscopic, photochemical, biochemical, immunochemical, electrical, optical or chemical means.

Accordingly, virtually any label that produces a detectable, quantifiable signal and that is capable of being attached to a nucleotide and incorporated into the generated amplicon, can be used in conjunction with the methods of the invention. Suitable labels include, by way of example and not limitation, radioisotopes, fluorophores, chromophores, chemiluminescent moieties, chemical labelling such as ULS labelling (Universal Linkage system; Kreatech) and ASAP (Accurate, Sensitive and Precise; Perkin Elmer), etc. Suitable labels may induce a colour reaction and/or may be capable of bio-, chemi- or photoluminescence.

Means for detecting such labels are well known to those of skill in the art. Thus, for example, radiolabels may be detected using photographic film or scintillation counters, fluorescent markers may be detected using a photodetector to detect emitted illumination. Enzymatic labels are typically detected by providing the enzyme with an enzyme substrate and detecting the reaction product produced by the action of the enzyme on the substrate; colorimetric labels are detected by simply visualizing the coloured label, and chemical labels by for example a platinum group form coordinative bonds with the labelling target, firmly coupling the label to the target.

Preferably, the position of the label will not interfere with generation, hybridisation, detection or other post-hybridisation modification of the labelled polynucleotide. A variety of different protocols may be used to generate the labelled nucleic acids, as is known in the art, where such methods typically rely on the labelled primers, or enzymatic generation of labelled nucleic acid using a labelled nucleotide. For instance, label may be incorporated into a nucleic acid during amplification steps in order to produce labelled amplicons. Alternatively, the generated amplicons may be labelled after the amplification.

A variety of labels may be employed, where such labels include fluorescent labels, isotopic labels, enzymatic labels, chemical labels, electron-dense reagents, particulate labels, etc. For example, suitable isotopic labels include radioactive labels, e.g. ³²P, ³³P, ³⁵S, ³H, ¹²⁵I, ¹⁴C. For example, suitable enzymatic labels include glucose oxidase, peroxidase, uricase, alkaline phosphatase etc. Other suitable labels include size particles that possess light scattering.

Fluorescent labels are particularly suitable because they provide very strong signals with low background. Fluorescent labels are also optically detectable at high resolution and quick scanning procedure. Fluorescent labels offer the additional advantage that irradiation of a fluorescent label with light can produce a plurality of emissions. Thus, a single label can provide for a plurality of measurable events.

Accordingly, in a further embodiment of the present invention, a method for target nucleic acid detection and quantification in an intact genomic DNA sample is provided, wherein the amplified probes are provided with a fluorescent label.

Desirably, fluorescent labels should absorb light above about 300 nm, usually above about 350 nm, and more usually above about 400 nm, usually emitting at wavelengths greater than about 10 nm higher than the wavelength of the light absorbed.

Particular useful fluorescent labels include, by way of example and not limitation, fluorescein isothiocyanate (FITC), rhodamine, malachite green, Oregon green, Texas Red, Congo red, SybrGreen, phycoerythrin, allophycocyanin, 6-carboxyfluorescein (6-FAM), 2′,7′-dimethoxy-4′,5′-dichloro-6-carboxyfluorescein (JOE), 6-carboxy X-rhodamine (ROX), 6-carboxy-2′,4′,7′,4,7-hexachlorofluorescein (HEX), 5-carboxyfluorescein (5-FAM), N,N,N′,N′-tetramethyl-6-carboxyrhodamine (TAMRA), cyanine dyes (e.g. Cy5, Cy3), BODIPY dyes (e.g. BODIPY 630/650, Alexa542, etc.), green fluorescent protein (GFP), blue fluorescent protein (BFP), yellow fluorescent protein (YFP), red fluorescent protein (RFP), and the like, (see, e.g., Molecular Probes, Eugene, Oreg., USA).

In one embodiment, the use of a method according to the present invention and as described herein is provided for detection and quantification of target nucleic acids in an intact genomic DNA sample.

Yet another object of the present invention is to provide for a method for target nucleic acid detection and quantification in an intact genomic DNA sample comprising the steps of: (a) providing intact genomic DNA and denaturing said genomic DNA; (b) performing a hybridisation according to a method as described within the present specification; (c) recovering hybridised probes; and essentially simultaneously amplifying any recovered probe using a single primer pair, each member of said primer pair binding to each recovered probe onto the respective flanking primer binding sequences of said probe; and (d) qualitatively and quantitatively analysing the recovered amplified probes of step (c).

Recovered amplified probes may be analysed by gel electrophoresis. However, for improved reproducibility and accuracy of procedures, an automated system for determining genomic profiles is contemplated. In particular, the present invention connotes the use of a probe array or microarray which is interrogated with the amplified hybridised identifier probes provided by the methods of the invention. The term “probe array” relates to a substrate having a high density matrix pattern of positionally defined specific recognition reagents. The multiple probe copies provided by the method of the invention are capable of interacting, e.g. hybridising, with their specific counterparts, i.e. the specific recognition reagents, on the array. Because the specific recognition reagents are positionally defined, the sites of interaction will define the specificity of each interaction. The specific recognition reagents will typically be deoxyribonucleotide (DNA) probes, in which case said probe array is known as an oligonucleotide or cDNA array. Various array production methods are known in the art.

Accordingly, in a further embodiment of the present invention, a method for target nucleic acid detection and quantification in an intact genomic DNA sample is provided, wherein the analysis of the recovered amplified probes is by microarray analysis.

The term “nucleic acid” as used herein means a polymer composed of nucleotides, e.g. deoxyribonucleotides or ribonucleotides. The terms “deoxyribonucleic acid” and “DNA” as used herein means a polymer composed of deoxyribonucleotides. The term “oligonucleotide” as used herein denotes single stranded nucleotide multimers of from about 10 to about 100 nucleotides in length. The term “polynucleotide” as used herein refers to single or double stranded polymer composed of nucleotide monomers of from about 10 to about 5000 nucleotides in length, usually of greater than about 100 nucleotides in length up to about 1000 nucleotides in length.

For a given substrate size, the upper limit is determined only by the ability to create and detect the spots in the array. The preferred number of spots on an array generally depends on the particular use to which the array is to be put. For example, mutation detection may require only a small array. In general, arrays contain from 2 to about 10⁶ spots, or from about 4 to about 10⁵ spots, or from about 8 to about 10⁴ spots, or between about 10 and about 2000 spots, or from about 20 to about 200 spots.

Suitable arrays may be of any desired size, from two spots to 10⁶ spots or even more. The upper and lower limits on the size of the substrate are determined solely by the practical considerations of working with extremely small or large substrates.

The immobilized polynucleotides may be as few as four, or as many as hundreds, or even more, nucleotides in length. Contemplated, as polynucleotides according to the invention are nucleic acids that are typically referred to in the art as oligonucleotides and also those referred to as nucleic acids. Thus, the arrays within the present invention are useful in applications where the generated identifier probe copies are hybridised to immobilized arrays of relatively short (such as, for example, having a length of approximately 6, 8, 10, 20, 40, 60, 80, or 100 nucleotides) detector probes.

The detector polynucleotides can be immobilized on the substrate using a wide variety of techniques. For example, the polynucleotides can be adsorbed or otherwise non-covalently associated with the substrate (for example, immobilization to nylon or nitrocellulose filters using standard techniques); they may be covalently attached to the substrate; or their association may be mediated by specific binding pairs, such as biotin and streptavidin.

A number of materials suitable for use as substrates for microarray analysis purposes in the instant invention have been described in the art. Exemplary suitable materials include, for example, acrylic, styrene-methyl methacrylate copolymers, ethylene/acrylic acid, acrylonitrile-butadienestyrene (ABS), ABS/polycarbonate, ABS/polysulfone, ABS/polyvinyl chloride, ethylene propylene, ethylene vinyl acetate (EVA), nitrocellulose, nylons (including nylon 6, nylon 6/6, nylon 6/6-6, nylon 6/9, nylon 6/10, nylon 6/12, nylon 11 and nylon 12), polycarylonitrile (PAN), polyacrylate, polycarbonate, polybutylene terephthalate (PBT), polyethylene terephthalate (PET), polyethylene (including low density, linear low density, high density, cross-linked and ultra-high molecular weight grades), polypropylene homopolymer, polypropylene copolymers, polystyrene (including general purpose and high impact grades), polytetrafluoroethylene (PTFE), fluorinated ethylene-propylene (FEP), ethylene-tetrafluoroethylene (ETFE), perfluoroalkoxyethylene (PFA), polyvinyl fluoride (PVF), polyvinylidene fluoride (PVDF), polychlorotrifluoroethylene (PCTFE), polyethylene-chlorotrifluoro-ethylene (ECTFE), polyvinyl alcohol (PVA), silicon styreneacrylonitrile (SAN), styrene maleic anhydride (SMA), and glass.

Other exemplary suitable materials for use as substrates in the arrays of the present invention include metal oxides. Metal oxides provide a substrate having both a high channel density and a high porosity, allowing high density arrays comprising different specific recognition reagents per unit of the surface for sample application. In addition, metal oxides are highly transparent for visible light. Metal oxides are relatively cheap substrates that do not require the use of any typical microfabrication technology and, that offers an improved control over the liquid distribution over the surface of the substrate, such as electrochemically manufactured metal oxide membrane. Metal oxide membranes having through-going, oriented channels can be manufactured through electrochemical etching of a metal sheet. Metal oxides considered are, among others, oxides of tantalum, titanium, and aluminium, as well as alloys of two or more metal oxides and doped metal oxides and alloys containing metal oxides. The metal oxide membranes are transparent, especially if wet, which allows for assays using various optical techniques. Such membranes have oriented through-going channels with well-controlled diameter and useful chemical surface properties. Patent application EP-A-0 975 427 is exemplary in this respect, and is specifically incorporated in the present invention.

As will be appreciated in the art, methods according to the present invention find particular use in methods for genomic screening and gene expression studies. For example, intact genomic DNA or RNA can be immobilized onto a matrix as described herein and flow-through hybridised with for example two antisense oligonucleotides which leave a 10 base gap between them upon hybridisation onto a target sequence. By use of DNA polymerase in the presence of the necessary dNTPs, said gap will be filled which may than subsequently ligated. Un-hybridised probes are then flow-through washed off through said matrix after which the hybridised oligonucleotides are eluted, quantitatively amplified and analysed by means of a microarray. As another example, intact genomic DNA or RNA can be immobilized onto a matrix as described herein and flow-through hybridised with for example short PCR-amplified probes.

It is another object of the invention to provide for the use of a method according to the present specification and as described herein for genomic screening.

By the term “genomic screening” is meant the screening for genetic variability within for example a genetic locus. Mutations may be located within genes for a variety of scenarios: e.g., for detecting sequence changes of HIV mutants which generate drug resistance and for detecting sequence changes of genes in relation to cancer development. Said sequence changes may include for example sequence deletions and sequence duplications.

Accordingly, in a further embodiment, the use of a method according to the present invention and as described herein is provided for detecting deletions or duplications in genomic DNA.

In a further embodiment, the use of a method according to the present invention and as described herein is provided for genome profiling.

By the term “genome profiling” is meant the identification of the presence and/or absence of genomic differences or variation between genomes of closely related species such as for example between humans and other primates. Genome profiling encompasses the identification of species using genotypes (genotyping).

In a further embodiment, the use of a method according to the present invention and as described herein is provided for identifying and quantitatively detecting the degree of pathogenesis, disease or contamination in a sample.

In a further embodiment, the use of a method according to the present invention and as described herein is provided for identifying and detecting the presence of infectious agents in a sample.

In a further embodiment, the use of a method according to the present invention and as described herein is provided for genotyping pathogens present in a sample.

The present invention also provides kits for performing the subject flow-through hybridisation methods. The subject kits at least include a device for flow-through hybridisation of probes onto immobilized intact genomic DNA comprising a well holder, said well holder comprising one or more round wells with a fixed diameter, said wells exposing a fibre network matrix.

Accordingly, it is a further object of the present invention to provide a kit for flow-through hybridisation of probes onto immobilized intact genomic DNA comprising a device for flow-through hybridisation according as described in the present specification and instructions to carry out a method according to the specifications as described herein.

Kits may further comprise one or more reagents employed in the various methods, such as amplification primers for generating amplicons of the hybridised identifier probes as well as the amplification components. As used herein, the term “amplification components” refers to the reaction reagents such as enzymes, buffers, and nucleic acids including nucleotides necessary to perform an amplification reaction to form amplicons or amplification products of the hybridised identifier probes. A primer is a nucleic acid molecule with a 3′ terminus that is either “blocked” and cannot be covalently linked to additional nucleic acids or that is not blocked and possesses a chemical group at the 3′ terminus that will allow extension of the nucleic acid chain such as catalysed by a DNA polymerase or reverse transcriptase.

Accordingly, in one embodiment of the present invention a kit for target nucleic acid detection in an intact genomic DNA sample is provided additionally comprising (a) a set of probes, wherein each probe is flanked 5′ and 3′ by primer binding regions with said 5′ and 3′ flanking primer binding sequences being the same or substantially the same for each probe; (b) a single primer pair, each member of said pair being complementary to a primer binding region; (c) optionally amplification components allowing the amplification of any recovered hybridised probe; and (d) optionally a microarray, said microarray allowing analysis of the hybridisation results obtained by a method as described within the present specification.

### SHORT DESCRIPTION OF THE FIGURES

FIG. 1 shows a schematic representation of the hybridisation method of the present invention. Intact genomic material is first immobilized onto a suitable matrix such that said genomic material becomes permeated within said matrix material (step 1). A set of identifier probes is subsequently hybridised by flow-through onto the immobilized intact genomic material (step 2) to arrive at the formation of hybridised intact genomic DNA/probe complexes (step 3). Unbound probes are washed off by flow-through washing (step 4), leaving said formed hybridised intact genomic DNA/probe complexes (step 5) for further analysis (step 6).

FIG. 2 illustrates the electrophoresis results of the PCR products corresponding to 12 control probes as prepared according to the description in Example 1, paragraph 1.1.1. Probe numbers, probe names, probe sizes and genomic locations are as mentioned in Table 1. M, 100 bp DNA ladder (Cat. No. 15628-050; invitrogen).

FIG. 3 illustrates the electrophoresis results of the PCR products corresponding to the MSH2 probes that were prepared from direct amplification of human genomic DNA using 18 specific primer pairs flanked by the same sequences as PZA and PZB as described in Example 1, paragraph 1.1.2. The sequences of the 18 specific MSH2 primer pairs for PCR amplification are as mentioned in Table 2.

M, 100 bp DNA ladder; Q, negative control.

FIG. 3A illustrates said results after PCR in PCR mixture without DMSO;

FIG. 3B illustrates said results after PCR in PCR mixture with DMSO.

FIG. 4 illustrates the electrophoresis results of the PCR products after flow-through hybridisation of 1 μg of immobilized intact genomic DNA from healthy control individual 1 with a control probe mixture in formamide hybridisation solution as described in Example 1, paragraph 1.3.

a, first PCR; b, repeat PCR reaction from the same multiplex amplifiable probe hybridisation solution; M, 100 bp DNA ladder; P, PCR from control probe mix; Q, negative control for PCR; W, water control for hybridisation; 1, intact genomic DNA from healthy control individual 1. No PCR products were obtained from the negative controls from PCR and hybridisation.

FIG. 5 illustrates the electrophoresis results of the PCR products after flow-through hybridisation of 250 ng of immobilized intact genomic DNA from three healthy control individuals (individuals 1, 4, and 5).

M, 100 bp DNA ladder; P, PCR from control probe mix; Q, negative control for PCR; W, water control for hybridisation. No PCR products were obtained from the negative controls from PCR and hybridisation.

FIG. 6 illustrates the quality control of amplified probes as described in Examples 1 and 2.

FIG. 6_{A-C} illustrates gel-electrophoresis results of the quality control of 12 control probes, 18 MSH2 probes and 19 MLH1 probes.

FIG. 7 illustrates the quality of the PCR products after flow-through hybridisation of 1 μg of immobilized intact genomic DNA from healthy control individual 5 as described in Example 2.

M, 100 bp DNA ladder; Q, negative control for PCR without hybridisation; W, PCR product obtained from water control after flow-through hybridisation and post-washes on Nylon membrane; 5, PCR product obtained from intact genomic DNA of individual 5 after flow-through hybridisation and post-washes on the Nylon membrane.

FIG. 8 illustrates the quality of the PCR products after flow-through hybridisation of 1 μg of immobilized intact genomic DNA from healthy control individual 5 as described in Example 3.

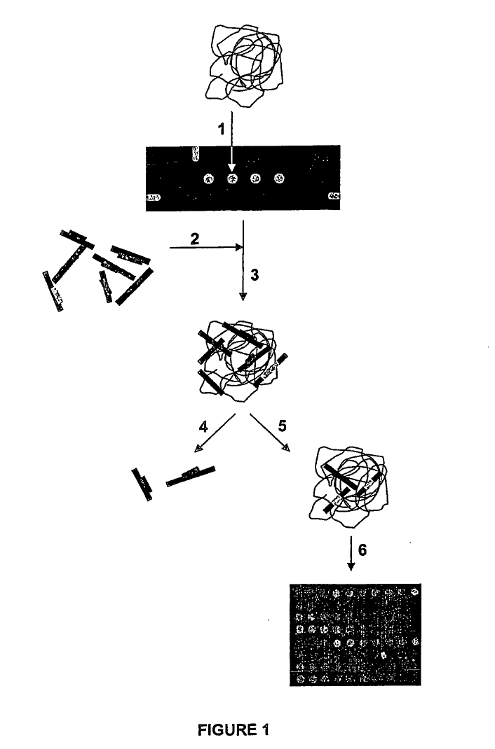

M, 100 bp DNA ladder; Q, negative control for PCR without hybridisation; 5a, PCR product directly obtained from the intact genomic DNA of control individual 5 without hybridisation; W, PCR product obtained from water control after flow-through hybridisation and post-washes on Anodisc 25 membrane; 5b, PCR product obtained from the intact genomic DNA of control individual 5 after flow-through hybridisation and post-washes on Anodisc 25 membrane; P, PCR product directly obtained from PMPP probe mix without hybridisation.

### EXAMPLES

The following examples of the invention are exemplary and should not be taken as in any way limiting.

### Example 1

### Flow-Through Hybridisation of Intact Genomic DNA on Whatman 3MM Chr Paper

1.1 Probe Preparation

1.1.1 Probe Preparation—Control Probes

Plasmids (100 ng/μl) were obtained from the Institute of Genetics, University of Nottingham, Queen's Medical Centre, Nottingham NG7 2UH, UK (Dr. John Armour). Probe DNA was amplified from these plasmids using flanking vector primers PZA

(AGTAACGGCCGCCAGTGTGCTG; SEQ ID No. 1) and PZB
(CGAGCGGCCGCCAGTGTGATG; SEQ ID No. 2) (Isogene).

The PCR reaction was performed (PTC-200 Peltier Thermal Cycler; MJ Research INC; Massachusetts, USA) in a reaction mixture comprising 5 μl 10× PCR gold buffer (PE) (Cat. No. 4311816; Applied Biosystems), 2.5 μl MgCl₂ (25 mM), 1.25 μl dNTPs (10 mM; Amersham Pharmacia Biotech), 0.125 μl AmpliTaq Gold® (5 U/μl) (Cat. No. 4311816; Applied Biosystems), 1 μl PZA forward primer (10 pM), 1 μl PZB reverse primer (10 pM), 1 μl plasmid DNA, and 38.125 μl HLPC-water. A PCR program with following cycle order was completed: cycle 1, 3 min at 94° C.; cycles 2 to 35, 1 min at 94° C., 1 min at 60° C., 1 min at 72° C.; and finally 10 min at 72° C.

Obtained PCR products were purified using the Qiaquick PCR purification kit (Cat. No. 28106, QIAGEN, Germany) and dissolved in 300 μl EB buffer (10 mM Tris-Cl, pH8.5).

A 40-times dilution was made up from the purified PCR products (5 μl PCR products+195 μl water) for DNA concentration measurement using a SpectramaxPlus 384 reader (Molecular Devices; Sunyvale, Calif., USA). The following concentrations were obtained: probe 1, 27 ng/μl; probe 2, 26 ng/μl; probe 3, 23 ng/μl; probe 4, 11 ng/μl; probe 5, 5 ng/μl; probe 6, 12 ng/μl; probe 7, 13 ng/μl; probe 8, 13 ng/μl; probe 9, 19 ng/μl; probe 10, 6 ng/μl; probe 11, 22 ng/μl and probe 12, 5 ng/μl.

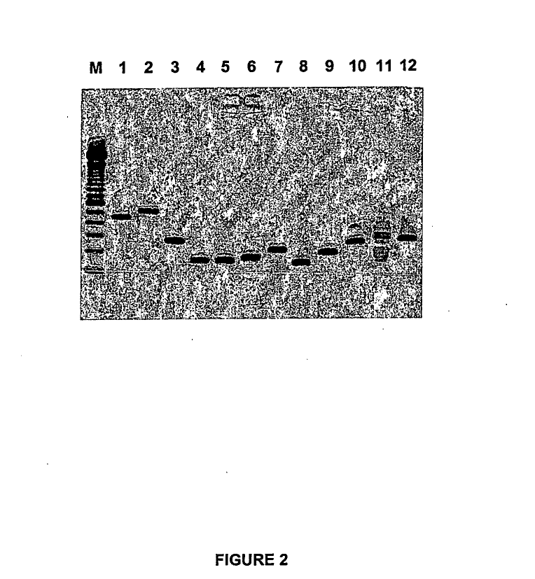

Subsequently, 10 μl PCR products were loaded on a 2% agarose gel in 400 mM Tris-Acetate/10 mM EDTA (TAE) (0.5×; Cat. No. 15558-042; GIBCOBRL). Electrophoresis was performed at 100V for 40 min (FIG. 2).

Probe names, sizes and sequences are given in Table 1.

1.1.2 Probe Preparation—MSH2 Probes

Genomic DNA 1 was obtained from a healthy control individual via the blood bank of the University Hospital of Leiden, The Netherlands. MSH2 probes were prepared by PCR on genomic DNA 1. The MSH2 primers used are shown in Table 2.

PCR was performed in a reaction mixture comprising 5 μl PCR Gold buffer (10×), 2.5 μl MgCl₂ (25 mM), 1.25 μl dNTPs (10 mM), 0.125 μl AmpliTaq Gold (5 U/μl), 1 μl MSH2 forward primer (10 pM), 1 μl MSH2 reverse primer (10 pM), 1 μl genomic DNA (100 ng/μl), and 38.125 μl HLPC-water.

Additionally, a second PCR mixture was made up including 5 μl DMSO.

A PCR program with following cycle order was completed: cycle 1, 3 min at 94° C.; cycles 2 to 5, 1 min at 94° C., 1 min at 56° C., 1 min at 72° C.; cycles 6 to 10, 1 min at 94° C., 1 min at 53° C., 1 min at 72° C.; cycles 11 to 35, 1 min at 94° C., 1 min at 50° C., 1 min at 72° C.; and finally 10 min at 72° C.

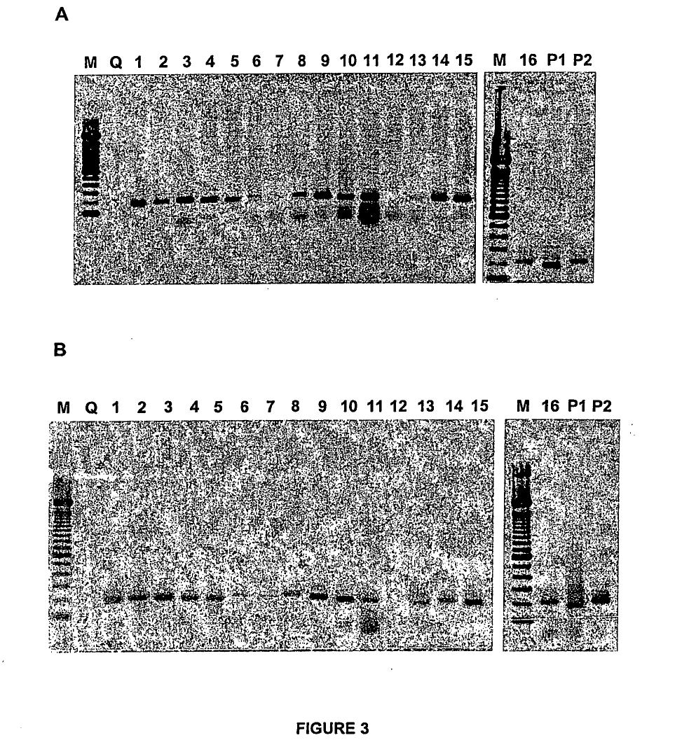

Obtained PCR products were purified using the Qiaquick PCR purification kit and dissolved in 300 μl EB buffer (10 mM Tris-Cl, pH 8.5). A 40-times dilution was made up from the purified PCR products (5 μl PCR products+195 μl water) for DNA concentration measurement using a SpectramaxPlus reader. Subsequently, 10 μl PCR products were loaded on a 2% agarose gel in TAE (0.5×). Electrophoresis was performed at 100V for 40 min (FIG. 3).

1.2 Poly-L-lysine (PLL) Coating of Whatman 3MM Chr Paper

Whatman 3MM Chr paper (cat No. 3030 917) was cut into small pieces and 50 of them were placed in a Teflon holder (see WO 02/072268 for further specifications on said holder). A 0.01% poly-L-lysine solution was prepared with 35 ml Poly-L-lysine (0.1%, Sigma; Cat. No. P 8920), 35 ml PBS (1×), and 280 ml filtered HLPC water. This 350 ml PLL solution was subsequently poured in a 600 ml beaker which was placed on a plate shaker. The holder was gently moved up and down to prevent that air bubbles would be enclosed between the holder and the Whatman papers. The beaker was closed with parafilm and incubated on the plate shaker at room temperature with shaking at 100 rpm for 1 hour. Subsequently, Whatman papers were transferred to a second beaker filled with 350 ml HLPC-water; the holder was again moved gently up and down for a couple of times. This transfer was repeated at least one more time. Papers were than transferred to an aluminium foil dish and placed for 2 hours in a vacuum oven at 37° C. under vacuum. After turning off the vacuum pump, papers were allowed to cool down to room temperature after which they were stored in a dark and dry place.

1.3 Intact Genomic DNA Hybridisation on Whatman 3MM Chr Paper Coated with PLL Whereby Washing is Performed with a Reduced Washing Volume

1.3.3. Solutions

50% deionised formamide hybridisation solution was prepared by mixing together 5 ml formamide (100% deionised), 1.5 ml SSC (20×), 0.5 ml SDS (20%), 1 ml Denhardts solution (100×), 20 μl Tween-20, and 1.8 ml HLPC-water. Pre-hybridisation buffer was prepared by drying 10 μl herring sperm DNA (10 μg/μl) using a SpeedVac for 15 min and adding 50 μl of hybridisation buffer to this dried herring sperm DNA. Probe mixture was prepared by mixing 4 μl control probe mix (10 ng/each probe), 20 μl Cot-1 DNA, and 1 ul blocker (50 pmol/μl) of PZAX (AGTAACGGCCGCCAGTGTGCTGGAATTCTGCAGAT; SEQ ID No. 3)/PZBX (CGAGCGGCCGCCAGTGTGATGGA; SEQ ID No. 4), Cot-1 DNA (Cat. No. 1581074; Roche) was used to block repetitive sequences of human genomic DNA for specific hybridisation. The probe mix was dried using a SC110A-240 SpeedVac Plus (Savant Instrument INC; New York, USA) for 15 min after which 50 μl of hybridisation buffer was added.

1.3.2 Intact Genomic DNA Hybridisation on Whatman 3MM Chr Paper Coated with PLL

A PLL-coated Whatman paper slide was made onto which 1 μg denatured intact genomic DNA of control 1 was spotted. Water was spotted as control on an individual paper. The DNA was allowed to dry for 10 min at room temperature. The DNA was subsequently UV cross-linked (UV Stratalinker 1800; Cat. No. 400072; Stratagene; Calif., USA) to the Whatman papers at 50 mJ on both sides. 50% deionised formamide hybridisation solution was prepared by mixing together 5 ml formamide (100% deionised), 1.5 ml SSC (20×), 0.5 ml SDS (20%), 1 ml Denhardts solution (100×), 20 μl Tween-20, and 1.8 ml HLPC-water. The pre-hybridisation solution consisting of 100 μg herring sperm DNA (10 μg/μl; Cat. No. D1816; Promega) in hybridisation solution was boiled for 5 min and placed on ice. The Whatman papers were pre-hybridised in 50 μl pre-hybridisation solution at 42° C. for 2 hours using flow-through system (0.2 bar pressure at 42° C. for 2 hours at 2 cycles/min). The pre-hybridisation solution was removed followed by a washing step once with hybridisation buffer.

The hybridisation probe mix was boiled for 5 min and placed on ice. 50 μl hybridisation probe mix solution was added and incubated at 42° C. for 4 hours using the flow-through system.

1.3.3 Post-Hybridisation Washes

The hybridisation mix was pipetted off and the Whatman papers were washed using flow-through system at 42° C. using 25 ml of solution 1 for 2.5 min and 25 ml of solution 2 for 2.5 min. Wash solution 1 consisted of 1% SSC (20×SSC, 3M NaCl, 0.3M Sodium Citrate) and 1% SDS and wash solution 2 consisted of 0.1% SSC and 0.1% SDS.

Individual Whatman papers were transferred into 50 μl of PCR buffer (1×) in 1.5 ml tubes and each boiled for 5 min. 5 μl of the boiled solution was transferred into a tube with PCR mixture comprising 5 μl PCR gold buffer (10×), 2.5 μl MgCl₂ (25 mM), 1.25 μl dNTPs (10 mM), 0.125 μl AmpliTaq Gold (5 U/μl), 1 μl PZA Forward primer (10 pM), 1 μl PZB reverse primer (10 pM), 5 μl sample solution, and 34.125 μl HLPC-water.

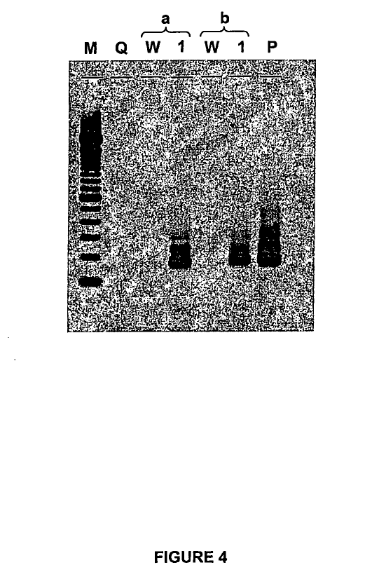

A PCR program with following cycle order was completed: cycle 1, 3 min at 94° C.; cycles 2 to 35, 1 min at 94° C., 1 min at 60° C., 2 min at 72° C.; and finally 10 min at 72° C. 10 μl PCR products were loaded onto a 2% agarose gel with TAE (0.5×). Electrophoresis was performed at 100V for 40 min. The results are shown in FIG. 4.

In conclusion, the above experiment demonstrates that Multiplex Amplification Probe Hybridisation can be performed by flow-through hybridisation on intact genomic DNA immobilized within a matrix or membrane structure.

1.4 Intact Genomic DNA Hybridisation on Whatman 3MM Chr Paper Coated with PLL Using Control Probes

1.4.1 Solutions

50% deionised formamide hybridisation solution was prepared by mixing together 5 ml formamide (100% deionised), 1.5 ml SSC (20×), 0.5 ml SDS (20%), 1 ml Denhardts solution (100×), 20 μl Tween-20, and 1.8 ml HLPC-water. Pre-hybridisation buffer was prepared by drying 10 μl herring sperm DNA (10 μg/μl) using a SpeedVac for 15 min and adding 30 μl of hybridisation buffer to this dried herring sperm DNA. Probe mixture was prepared by mixing 4 μl PMP22 control probe mix (10 ng/each probe, see Table 1), 20 μl Cot-1 DNA (1 mg/ml), and 1 μp PZAX/PZBX blocker (50 pmol/μl). The end-blocking primers PZAX and PZBX as described in Example 1, paragraph 1.3 (Isogene) were added to prevent cross-hybridisation between different probes used in the same mixture. The probe mix was dried using a SpeedVac for 15 min after which 30 μl of hybridisation buffer was added.

1.4.2 Intact Genomic DNA Hybridisation on Whatman 3MM Chr Paper Coated with PLL

250 ng of denatured intact genomic DNA of control individuals 1, 4 and 5 was spotted onto individual PLL-coated paper. Water was spotted as control on an individual paper.

The DNA was allowed to dry for 10 min at room temperature. The DNA was subsequently UV cross-linked to the Whatman papers at 50 mJ on both sides. The pre-hybridisation solution was boiled for 5 min and placed on ice. Whatman papers ware pre-hybridised in 30 μl pre-hybridisation solution at 42° C. for 2 hours using flow-through system (at 0.2 bar and 2 cycles/min). The pre-hybridisation solution was removed followed by a washing step once with hybridisation buffer.

The hybridisation probe mix was boiled for 5 min and placed on ice. 30 μl hybridisation probe mix solution was added and incubated at 42° C. for 4 hours using the flow-through system.

1.4.3 Post-Hybridisation Washes

Wash solutions were prepared as before.

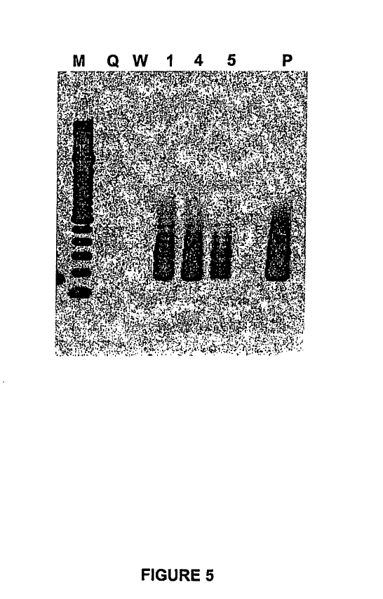

The hybridisation mix was pipetted off and the Whatman papers were washed using flow-through system at 65° C. using 50 ml of solutions and 50 ml of solution 2 for 10 min. Individual Whatman papers were transferred into 50 μl of PCR buffer (1×) in 1.5 ml tubes and each boiled for 5 min. 5 μl of the boiled solution was transferred into a tube with PCR mixture comprising 5 μl PCR gold buffer (10×), 3 μl MgCl2 (25 mM), 5 μl dNTPs (2.5 mM), 0.125 μl AmpliTaq Gold (5 U/μl), 1 μl PZA Forward primer (10 pM), 1 μl PZB reverse primer (10 pM), 5 μl sample solution, and 22.375 μl HLPC-water. A PCR program with following cycle order was completed: cycle 1, 3 min at 94° C.; cycles 2 to 35, 1 min at 94° C., 1 min at 60° C., 2 min at 72° C.; and finally 10 min at 72° C. 10 μl PCR products were loaded onto a 2% agarose gel with TAE (0.5×). Electrophoresis was performed at 100V for 40 min. The results are shown in FIG. 5.

In conclusion, the above experiment demonstrates that strong PCR bands were obtained upon electrophoresis of PCR products after flow-through hybridisation of a small amount of intact genomic DNA from individuals 1, 4 and 5 with the control probes.

### Example 2

### Flow-Through Hybridisation of Intact Genomic DNA on Nylon Membrane—0.45 μm Pore Diameter

In this experiment, Nylon membranes with 0.45 μm pore diameter (Amersham Biosciences, Cat No. RPN303B) were used to explore the use thereof in intact genomic DNA flow-through hybridisation.

2.1. Flow-Through Hybridisation of Intact Genomic DNA on Nylon Membrane

2.1.1. Hybridisation

1 μg of intact genomic DNA of control individual 5 and water were spotted on individual Nylon membranes. The DNA spot was dried for 10 minutes at room temperature. Subsequently the DNA was cross-linked to the filters at 50 mJ on both sides of the membranes. 2 ml of pre-hybridisation solution was prepared by adding together 280 μl 1M NaH₂PO₄, 720 μl Na₂HPO₄, 700 μl 20% SDS, 276 μl HLPC-water, 20 μl herring sperm DNA and 4 μl of 0.5M EDTA. The Nylon filters were pre-hybridised in 50 μl pre-hybridisation solution at 65° C. for 2 hours using flow-through system as described in U.S. Pat. No. 6,383,748 B1 (0.2 bar pressure at 42° C. for two hours at 2 cycles/min). The pre-hybridisation solution was removed and replaced with 50 μl of Cot-1 DNA solution and flown-through for 30 minutes at 65° C. The Cot-1 solution was subsequently removed and 50 μl hybridisation solution comprising the PMP22 control probes (see Table 1) was added. Flow-through hybridisation was performed at 65° C. during 4 hours.

2.1.2. Post-Hybridisation Washes

For the post-hybridisation washes wash solutions 1 and 2 were prepared and incubated at 65° C. Wash solution 1 was prepared by diluting 25 ml 20% SSC and 25 ml 20×SDS up to 500 ml with HLPC-water. Wash solution 2 was prepared by diluting 2.5 ml 20% SSC and 2.5 ml 20×SDS up to 500 ml with HLPC-water. The hybridisation mixture was pipetted off from the Nylon membranes after which these membranes were washed subsequently with 50 ml wash solution 1 and 50 ml wash solution 2 at 65° C. using flow-through system as described in U.S. Pat. No. 6,383,748 B1.

2.1.3. PCR

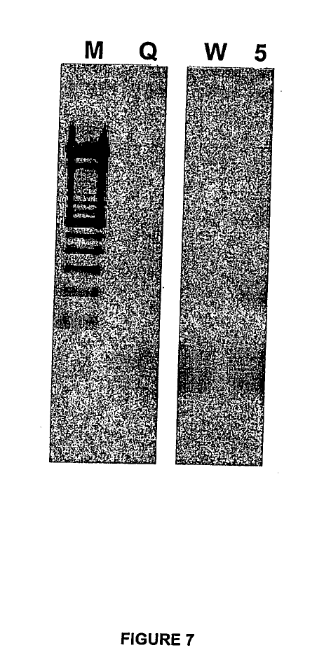

The individual Nylon membranes were transferred into 50 μl of HLPC-water in a 1.5 ml tube and boiled for 5 minutes. 2 μl of the boiled solution was then transferred into a new tube containing PCR reagents including 5 μl 10× PE buffer, 2.5 μl 25 mM MgCl₂, 1.25 μl 10 mM dNTP, 0.125 μl PE Taq (5 U/μl), 1 μl PZA forward primer (50 pM; see also Example 1), 1 μl PZB reverse primer (50 pM; see also Example 1)) and 37.125 μl HLPC-water. A PCR program with following cycle order was completed: cycle 1, 5 min at 94° C.; cycles 2 to 35, 45 sec at 94° C., 1 min at 57° C., 1 min at 68° C.; and finally 10 min at 68° C. 10 μl PCR products were loaded onto a 2% agarose gel with TAE (0.5×). Electrophoresis was performed at 100V for 35 min. The results are shown in FIG. 7. Only weak PCR results from both water and individual 5 samples were obtained indicating unspecific and inefficient hybridisation.

In conclusion, example 2 shows that a 0.45 μm diameter pore sized membrane does not allow efficient flow-through of the hybridising probes through the porous membrane.

### Example 3

### Flow-Through Hybridisation of Intact Genomic DNA on Anodisc 25

In this example, Multiple Amplification Probe Hybridisation (MAPH) was performed on intact genomic DNA immobilized onto Anodisc 25 membranes.

3.1. Intact Genomic DNA Hybridisation on Anodisc 25 (0.2 μm Pore Size)

3.1.1. Genomic Hybridisation

Individual Anodisc 25 membranes silanised with 3-mercaptopropyltrimethoxysilane (MPS) were spotted with respectively 1 μg denatured intact genomic DNA from control individual 5 and water. DNA was cross-linked to the membrane by UV cross linking at 50 mJ on both sides. Pre-hybridisation was carried out in 20 μl pre-hybridisation solution (see Example 3) at 65° C. during 30 minutes using PamGene's flow-through system (0.2 bar pressure at 42° C. for two hours at 2 cycles/min). The pre-hybridisation solution was removed and replaced with 20 μl of Cot-1 DNA solution and flown-through for 20 minutes at 65° C. The Cot-1 solution was removed and 20 μl hybridisation solution comprising PMP22 probes were added. Flow-through hybridisation was carried out at 65° C. for 1 hour.

3.1.2. Post-Hybridisation Washes

For the post-hybridisation washes wash solutions 1 and 2 were prepared and incubated at 65° C. Wash solution 1 was prepared by diluting 25 ml 20% SSC and 25 ml 20× SDS up to 500 ml with HLPC-water. Wash solution 2 was prepared by diluting 2.5 ml 20% SSC and 2.5 ml 20×SDS up to 500 ml with HLPC-water. The hybridisation mixture was pipetted off from the Anodisc 25-MPS membranes after which these membranes were transferred into two 1.5 ml tubes, one for the DNA sample and one for the water control. Membranes were washed subsequently with wash solution 1 for 30 minutes and with wash solution 2 for 45 minutes at 65° C.

3.1.3. PCR and Results

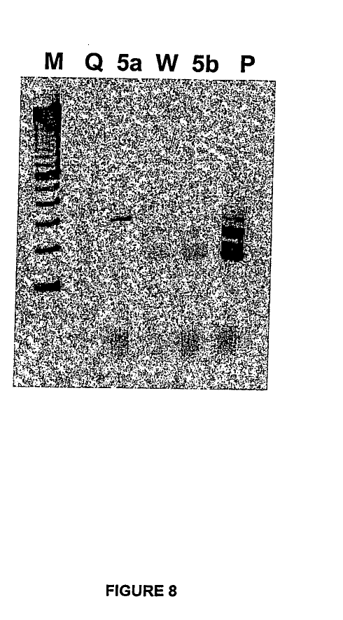

Individual washed membranes were transferred into 50 μl of 1× PCR buffer in 1.5 ml tubes and boiled for 5 minutes. 5 μl of the boiled solutions were subsequently transferred into 0.5 PCR thin-wall tubes. The following PCR mixture was added: 5 μl 10× PE buffer (Perkin Elmer), 2.5 μl 25 mM MgCl₂, 1.25 μl 10 mM dNTP, 0.125 μl PE Taq (5 U/μl), 1 μl PZA forward primer (50 pM), 1 μl PZB reverse primer (50 pM) and 34.125 μl HLPC-water. A PCR program with following cycle order was completed: cycle 1, 5 min at 94° C.; cycles 2 to 35, 45 sec at 94° C., 1 min at 57° C., 1 min at 68° C.; and finally 10 min at 68° C. 5 μl PCR products were loaded onto a 2% agarose gel with TAE (0.5×). Electrophoresis was performed at 100V for 45 min. The results are shown in FIG. 8. Only weak PCR results from both water and individual 5 samples were obtained indicating unspecific and inefficient hybridisation. This is due to the fact that that the small pore size did not allow the passing-through of the probes and an efficient post-hybridisation wash could not be established.

### Example 4

### Flow-Through Hybridisation of Intact Genomic DNA on Anodisc 25 Membrane—0.2 μm Pore Diameter (Whatman Plc.)

In this experiment, Anodisc 25 membranes (Whatman) were first positively charged by silanation with 3-aminopropyltriethoxysylane (APS). The purpose of the experiment was to evaluate the 0.2 μm-pore-size-membranes for use for intact genomic DNA flow-though hybridisation. Prior to hybridisation, the silanised membranes were blocked with either herring sperm DNA or with acetic anhydride and N,N-diisopropylethylamine.

4.1. Materials and Reagents

1. Anodisc 25, 0.2 μm membrane (Whatman)
2. 3-aminopropyltriethoxysylane (Acros, APS)
3. Acetic anhydride, N,N-diisopropylethylamine, DMSO, Dioxane, Acetonitril and dichloromethane
4. PMP22 probe mix (see Table 1)
5. genomic DNA from control individual 5
6. HLPC-water for negative control
7. Hybridisation reagents
   4.2. Silanation of Whatman Anodisc 25 Membranes with APS

A 1% APS solution was prepared by filtering 3 ml of APS and subsequently adding 2.5 ml filtered APS to 247.5 ml HLPC-water. A 600 ml beaker was filled with the 250 ml 1% APS solution and placed on a plate shaker. A number of 50 of Anodisc 25 membranes were placed in a Teflon holder (see WO 02/072268 for further specifications on said holder) and subsequently said holder was placed in the beaker containing the 1% APS solution. The holder was gently moved up and down to prevent that air bubbles would be enclosed between the holder and the Whatman papers. The beaker was dosed with parafilm and incubated on the plate shaker at room temperature with shaking at 100 rpm for 1 hour. Subsequently, Anodisc membranes were transferred to a second beaker filled with 250 ml HLPC-water; the holder was again moved gently up and down for a couple of times and finally kept in the HPLC solution for another 3 minutes. This transfer was repeated at least one more time. The Teflon holder was then transferred 2 times to 250 ml of 96% ethanol for 3 minutes. Membranes were then transferred to an aluminium foil dish and placed for 2 hours in a vacuum oven at 120° C. After turning off the vacuum pump, membranes were allowed to cool down to room temperature after which they were stored in a dark and dry place.

4.3. Intact Genomic DNA Hybridisation on Anodisc 25-APS Membranes Blocked with Herring Sperm DNA

1 μg denatured intact genomic DNA of control individual 5 and a water control were spotted onto individual Anodisc 25-APS membranes without UV cross-linking. The DNA was allowed to dry for 10 min at room temperature. 2 ml of pre-hybridisation solution was prepared by adding together 280 μl 1M NaH₂PO₄, 720 μl Na₂HPO₄, 700 μl 20% SDS, 276 μl HLPC-water, 20 μl herring sperm DNA and 4 μl of 0.5M EDTA. The spotted Anodisc-APS membrane was pre-hybridised in 50 μl pre-hybridisaton solution at 65° C. for 1 hour using PamGene's flow-through system (0.2 bar pressure at 42° C. for two hours at 2 cycles/min). The pre-hybridisation solution was removed and replaced with 50 μl of Cot-1 DNA solution and flown-through during 30 min at 65° C. The Cot-1 solution was subsequently removed and 50 μl of hybridisation solution comprising the PMP22 probes was added. Incubation was at 65° C. during 2 hours using flow-through. It appeared that the hybridisation was difficult but not impossible.

4.4. Intact Genomic DNA Hybridisation on Anodisc 25-APS Membranes Blocked with 0.5M Acetic Anhydride and 0.125M N,N-diisopropylethylamine

4.4.1. Solutions

10 ml of blocking solution was prepared by adding together 0.47 ml 0.5M acetic anhydride, 0.2175 ml 0.125M N,N-diisopropylethylamine and 9.3 ml dichloromethane.

500 ml post-hybridisation solution 1 was prepared by diluting 25 ml 20% SSC and 25 ml 20×SDS up to 500 ml in HLPC-water.

500 ml post-hybridisation solution 2 was prepared by diluting 2.5 ml 20% SSC and 2.5 ml 20×SDS up to 500 ml in HLPC-water.

4.4.2. Hybridisation Procedure

1 μg denatured intact genomic DNA of control individual 5 and a water control were spotted onto individual Anodisc 25-APS membranes without UV cross-linking. The DNA was allowed to dry for 10 min at room temperature.

The spotted membranes were placed with the lamination block on the washing system with vacuum (flow-through system). 20 μl of blocking solution was added to each sample and this step was repeated three more times. Samples were washed 4 times with 250 μl of 96% ethanol. During this step of the experiment, the membranes were damaged and hence no subsequent intact genomic DNA hybridisation could be performed on Anodisc-APS membranes blocked with acetic anhydride and N,N-diisopropylethylamine. The membranes could also not be washed using flow-through.

In conclusion, example 3 shows that hybridisation of intact genomic DNA immobilized onto Anodisc membranes with 2 μm pore diameter using flow-through resulted in either difficult hybridisation or damage of the membranes. Flow-through post-hybridisation washes were only possible for the water controls and not for the intact genomic DNA samples.

General Conclusion

From Examples 1 to 4 it was surprisingly found that only matrix pore sizes above 0.45 μm in diameter allow highly efficient flow-through analysis of intact genomic DNA.

Membranes with pore sizes of 0.2 μm (Example 4) and 0.45 μm (Example 3) showed difficulties for the intact non-manipulated genomic DNA to pass through those membranes and washing off non-hybridised probes through those membranes appeared to be impossible due to the small pore sizes.

The experiments showed that pore sizes>0.45 μm are required to assure efficient washing off of non-hybridised probes and hence highly specific hybridisations.

### Example 5

### Application of the Present Invention in the Analysis of Intact Genomic DNA Sample for Hereditary Nonpolyposis Colorectal Cancer

Colorectal cancer is one of the most common cancers in both men and women. The majority of the hereditary colorectal cancers are hereditary nonpolyposis colorectal cancer (HNPCC). HNPCC is an autosomal dominant disorder caused by mutations in mismatch repair (MMR) genes. The large majority of germline mutations detected in HNPCC families occur in the MSH2 and MLH1 genes. Approximately 25% of the germline mutations found in the MSH2 and MLH1 genes are large genomic deletions.

The present invention will allow identification of germline mutations in the MMR genes which will open the possibility of pre-symptomatic diagnosis in members of affected families. The results of a genetic screening will influence medical management of the patient or family members.

5.1 Preparation and Quality Control of the Probes for Microarray Production

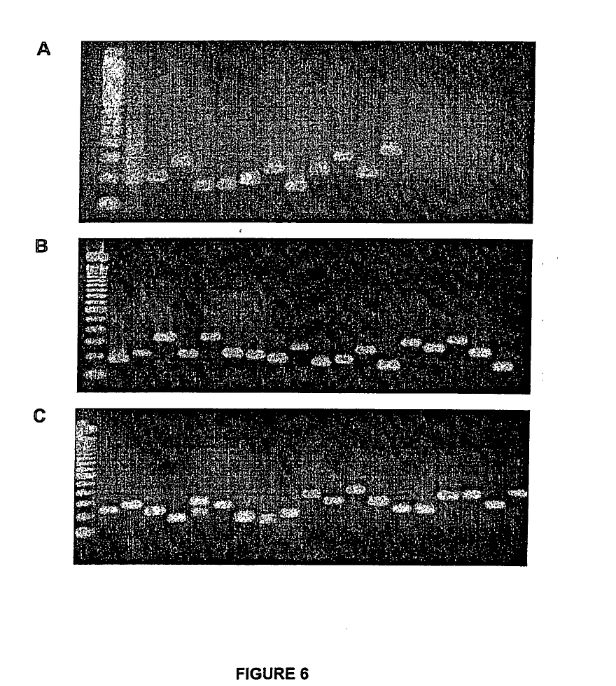

The probes of MSH2 P1 and P2 were generated from direct amplification of human genomic DNA prepared as described in Example 1. The probes (12 control probes, 16 MSH2 probes and 19 MLH1 probes) were generated from amplification of plasmids using the flanking vector primers PZA and PZB as described in Example 1. These plasmids were obtained from the Institute of Genetics, University of Nottingham, Queen's Medical Centre, Nottingham NG7 2UH, UK. The probes (12 control, 16 MSH2 and 19 MLH1) were prepared by cloning PCR products into the EcoRV site of pZero2 (Invitrogen). The sequences of the control probes that were cloned into the plasmids can be found in Table 1. The sequences of the MSH2 and MLH1 that were cloned into the plasmids can be found in Tables 3 and 4 respectively. Probe quality was monitored by gel electrophoresis onto a 2% agarose gel with TAE (0.5×) at 100V for 40 min of which the results are shown in FIGS. 6_{A-C}.

Control probes are used for normalization of microarray data and checking for PCR contamination. Human MSH2 is located on chromosome 2p21 and has 16 exons. The 18 MSH2 probes represent the 16 exons and two MSH2 promoter regions (P1 and P2). Human MLH1 is located on chromosome 3p21 and has 19 exons. The 19 MLH1 probes represent the 19 exons.

After flow-through hybridisation according to the present invention and post-hybridisation washes as described above, individual Whatman papers may be transferred in 50 μl of PCR buffer (1×) in 1.5 ml tubes and each boiled for 5 min. 5 μl of the boiled solution is then subsequently transferred into a tube with PCR mixture comprising 5 μl PCR gold buffer (10×), 3 μl MgCl₂ (25 mM), 5 μl dNTPs (2.5 mM), 0.125 μl AmpliTaq Gold (5 U/μl), 1 μl PZA forward primer (10 pM), 1 μl PZB reverse primer (10 pM), 5 μl sample solution and 22.375 μl HLPC water. A PCR program with following cycle order may be performed; cycle 1, 3 min at 94° C.; cycles 2 to 35, 1 min at 94° C., 1 min at 60° C., 2 min at 72° C.; and finally 10 min at 72° C.

During PCR amplification, labelled primer(s) or enzymatic generation of labelled nucleic acid may be used to generate labelled nucleic acids. The obtained labelled PCR products can then be purified using the Qiaquick purification kit and dissolved in 50 μl of EB buffer (10 mM Tris-Cl, pH 8.5) for further detection using a microarray.

Alternatively, chemical labels can be used (Kreatech) whereby for example a platinum group forms a coordinative bond with the labelling target, firmly coupling the label to the target.

For analysis of HNPCC patients, 49 of 60-mer oligo's (12 control, 18 MSH2 and 19 MLH1) having 40% to 50% GC content (Eurogentec) were selected for production of a microarray. The sequences of the oligo's can be found in Table 5. Upon manufacturing of a HNPCC microarray, clinical samples from patients with HNPCC can be analysed.

TABLE 1

SEQ ID Nos, names, sizes and genomic
location of the control probes

| | | |
|---|---|---|
| SEQ | | Probe |
| ID. | Probe | Size |

| | | | | |
|---|---|---|---|---|
| NO. | Name | (bp) | Location | Sequence |

| | | | | |
|---|---|---|---|---|
| 5 | 8D2 | 522 | Sub- | CTGCAGGGGCATCTCACGCCTC |
| | | | telomeric | CTGCCCTGGGGTTGGCCTGGGT |
| | | | (chrom. 19) | CTGGCAGAGATGGCCTCACCTC |
| | | | | TTTCCAGGACACAGCAGGTGGC |
| | | | | CACCAGCCCACAACCACTCTAC |
| | | | | ACAGCCACACGCGGTGTGAAGC |
| | | | | CACAGGGAAAGCATCCTAAGTG |
| | | | | ACCTCTGAAATCACCCTCAGCC |
| | | | | CCCTCTAGGACCCTCTGGGTAG |
| | | | | GGGTCGAGGACAGGTTCTCTGG |
| | | | | GCAACGGTTCCTGCCTCAGTCA |
| | | | | GGGGCTGGCTTTCTACTCCCGC |
| | | | | AACCAGGAGCCTGACTAACAAG |
| | | | | GGGATGGCAGGCGACCACATCA |
| | | | | AATGAGGCCCCAGTCTGGACGG |
| | | | | CAAGTTGACAACAGTCCCGCAG |
| | | | | GCTTGAGATCCACACCGAGTAA |
| | | | | CAGGGAGTTAACATATGGCCCC |
| | | | | AGGAGAGGTGGTCTGCAGGGTC |
| | | | | TGAGGAGGATCTGGCCCTTATA |
| | | | | TGACGTGCCATTTAACAGGAGA |
| | | | | G |
| 6 | 8B4 | 451 | Sub- | CTCTGGACTTTGCATATTTCAT |
| | | | telomeric | AATCCTGGCTCCAAAATACTAG |
| | | | (chrom. 19) | TGCAGGAGGATCCTAGGCCACG |
| | | | | TGGGGAATTATTTCTAAGGGTT |
| | | | | CCTGAAGTAACAATGGTAACAG |
| | | | | AGGTGAATTTTAGGAAGTAAAG |
| | | | | GATGTGAACTAGGAAAGAGATG |
| | | | | GACATAACTGAGGGGGGAAATG |
| | | | | ATACCCATGGGAACAGAGAAAC |
| | | | | CTGCGTGTGAGGTGTCAGCATG |
| | | | | AGGAGACCAGGGGCTCAAGTGA |
| | | | | GCCCCTCCGAGGGGATGGCTGT |
| | | | | GCTGCAGCAGAGATATGACTAG |
| | | | | AGACAACCCTCCTGGGCCGACT |
| | | | | GCTAGAGAACAGCAGCGCCACT |
| | | | | GTTGCGTCTCACTGTGTGGCTG |
| | | | | GGGAATAAAACGGCAGGAGGAG |
| | | | | GAGGGGACAGGAAACCAG |
| 7 | TBX5 | 332 | Sub- | gcttcttgtcctcagAGCAGAA |
| | B1 | | telomeric | CCTTGCGCGGGCACAGGGCCCT |
| | | | (chrom.12) | GGGCGCACCATGGCCGACGCAG |
| | | | | ACGAGGGCTTTGGCCTGGCGCA |
| | | | | CACGCCTCTGGAGCCTGACGCA |
| | | | | AAAGACCTGCCCTGCGATTCGA |
| | | | | AACCCGAGAGCGCGCTCGGGGC |
| | | | | CCCCAGCAAGTCCCCGTCGTCC |
| | | | | CCGCAGCCGCCTTCACCCAGCA |
| | | | | Ggtaaggagacctcgcgcttcg |
| | | | | ggtccctttgcagagatcaaag |
| | | | | tcagagtctggctttcctgctc |
| | | | | ggcttctcttg |
| 8 | 10B4 | 319 | Sub- | CTTGCACCCTGAGTCTGCAGTC |
| | D | | telomeric | GGCTGGGGTGGGGCCCCTGCAC |
| | | | (chrom. 7) | TGGAAGGAAGGCTGGGCCCCCG |
| | | | | ACCCAGTGGTGGCTTCAGGGCC |
| | | | | TGGTGAGGGTCACTTTCAGGCT |
| | | | | GCTTAGACCTGCAGCCTCCCCA |
| | | | | GGTGGTGTGCTCAGGCAGTGCC |
| | | | | TCTGGGCCGGCACAAACGGGAA |
| | | | | GGTGCTGAGGCCGCGTCTACCT |
| | | | | GTGGCTTTAAACACTGGCTTCC |
| | | | | ACCTGCTGGCCTCCGCGTCCCT |
| | | | | CCAGCCGGCCGCCTGGAG |
| 9 | TBX5 | 290 | TBX gene | cctggtgcgtgaactgaagcac |
| | Da | | (chrom. 12) | gcttcggtgcagtgcgctacct |
| | | | | ccagactctgagccaggcctct |
| | | | | agtacacctctccttcatctag |
| | | | | GTCTGTGACGGGCAAAGCTGAG |
| | | | | CCCGCCATGCCTGGCCGCCTGT |
| | | | | ACGTGCACCCAGACTCCCCCGC |
| | | | | CACCGGGGCGCATTGGATGAGG |
| | | | | CAGCTCGTCTCCTTCCAGAAAC |
| | | | | TCAAGCTCACCAACAACCACCT |
| | | | | GGACCCATTTGGGC |
| 10 | 5C3 | 282 | Sub- | CTGTGTTCCAGGTTCGGACTGG |
| | | | telomeric | GACCTGGAGGCGGCAGATGGGA |
| | | | (chrom. 8) | ACCTGTGCTCCCCTGACCTTAA |
| | | | | GAGCAGGGAGGTCAGAAGCCCT |
| | | | | GTGGGCTGAGTAATCCTCTGAA |
| | | | | GCACTTGCTGGCCTGGAAAGAA |
| | | | | TGTGTTTTTCAGGCTTAATCTG |
| | | | | TTATGATATGTTATCGGAAAAT |
| | | | | GTAATTTGCTGTGTAAACAAGC |
| | | | | AGCAGACTGGCCATTCTGCTGG |
| | | | | CAG |
| 11 | 4A4 | 235 | Sub- | TCTCTGCGTCCCCCTCCCCACA |
| | | | telomeric | CTCAGTTTCACTATGGCCAGAT |
| | | | (chrom. 4) | GCTGCCTCCGATGAAAGAAAGG |
| | | | | GAGCCCATGGCAGAGCGTCGAG |
| | | | | GGCGCCAGGGTGGCCACACAGG |
| | | | | CCAGGAGACCAACCTCTAACCC |
| | | | | TGATCTGACACAGGTCTAAGGG |
| | | | | GAAGGTCATGAAGAAGAAACAC |
| | | | | AG |
| 12 | A2Y | 222 | Y-linked | CTGTAACTCTAAGTATCAGTGT |
| | | | probe | gAAACGGGAGAAAACAGTAAAG |
| | | | (chrom. Y) | GCAACGTCCAGGAtAGAGTGAA |
| | | | | GCGACCCATGAACGCATTCATC |
| | | | | GTGTGGTCTCGCGATCAGAGGC |
| | | | | GCAAGATGGCT CTAGAGAATC |
| | | | | CCAGAATGCGAAACTCAGAGAT |
| | | | | CAGCAAGCAG |
| 13 | 11G9 | 188 | Sub- | CTGCAGTGGGATGAGATGGGCT |
| | | | telomeric | GAGGTTTGTGCCCCTGTAGCCG |
| | | | (chrom. 19) | TATGTGAACCATGGGGCAAGGT |
| | | | | GGTCAGCGGGGGTCAGAGGTAT |
| | | | | TGTACAAGGGTCCACATAGGAA |
| | | | | TGGCAGGGTGTGAGCAG |
| 14 | TBX5A2 | 170 | TBX gene | Tcaaagtcttcacctgagccct |
| | | | (chrom. 12) | gctctccagcgaggcgcactcc |
| | | | | tggcttttgcgctccaaagaag |
| | | | | aggtgggatagttggaggtgag |
| | | | | tttcaccctggaggactgaggg |
| | | | | g |
| 15 | 2D2 | 167 | X-linked | CCTCCCCTGCTCAGCACTCCTG |
| | | | probe | GGATTTGGAACCTCGTTCCTCT |
| | | | (chrom. X) | CTGCAAAGCCTCCTAGCCCGGT |
| | | | | TCTCCAGCCCTCCCCAGACCAA |
| | | | | TCATGGGATAGTGCCGTAGG |
| 16 | D11 | 153 | Non-human | CCACTACGTGAACCATCACCCT |
| | | | probe | AATCAAGTTTTTTGGGGTCGAG |
| | | | | GTGCCGTAAAGCACTAAATCGG |
| | | | | AACCCTAAAGGGAGCCCCCGAT |
| | | | | TTAGAG |

TABLE 2

Oligonucleotides used for direct
amplification of genomic DNA for generation
of MSH2 probes. MT, melting temperature;
AT, annealing temperature; GC, GC
content; P1, promoter region 1 of MSH2
gene; P2, promoter region 2 of MSH2 gene.
Values for size and GC-content (%)
indicated are of the amplified PCR
products using a pair of forward (F) and
reverse (R) primers.

| | | | | | | |
|---|---|---|---|---|---|---|
| | | | Size | MT | AT | GC |
| SEQ ID No. | exon | Primers, 5′→3′ | (bp) | (° C.) | (° C.) | (%) |

| | | | | | | |
|---|---|---|---|---|---|---|
| 17 | P1F | AGTAACGGCCGCCAGTGTGCTGGT | 189 | 66 | 56 | 57 |
| | | TTTCAATCTGTCGCCCAC | | | | |
| 18 | P1R | CGAGCGGCCGCCAGTGTGATGCCT | 189 | 66 | 56 | 57 |
| | | GGGCAACATGGTAAAAC | | | | |
| 19 | P2F | AGTAACGGCCGCCAGTGTGCTGCA | 218 | 68 | 58 | 45 |
| | | ATCTCCTGGGCTCAAGTG | | | | |
| 20 | P2R | CGAGCGGCCGCCAGTGTGATGACT | 218 | 62 | 52 | 45 |
| | | GCCTTTATTCTGCTTAC | | | | |
| 21 | 1F | AGTAACGGCCGCCAGTGTGCTGGC | 199 | 68 | 58 | 66 |
| | | TTCGTGCGCTTCTTTCAG | | | | |
| 22 | 1R | CGAGCGGCCGCCAGTGTGATGCCG | 199 | 70 | 60 | 66 |
| | | GCCCCATGTACTTGATC | | | | |
| 23 | 2F | AGTAACGGCCGCCAGTGTGCTGAA | 202 | 64 | 54 | 31 |
| | | GTCCAGCTAATACAGTGC | | | | |
| 24 | 2R | CGAGCGGCCGCCAGTGTGATGCAA | 202 | 64 | 64 | 31 |
| | | CTCTATACTGACGAACC | | | | |
| 25 | 3F | AGTAACGGCCGCCAGTGTGCTGGG | 208 | 62 | 52 | 45 |
| | | TAACAATGATATGTCAGC | | | | |
| 26 | 3R | CGAGCGGCCGCCAGTGTGATGGAG | 208 | 68 | 58 | 45 |
| | | GAGAGCCTCAAGATTGG | | | | |
| 27 | 4F | AGTAACGGCCGCCAGTGTGCTGAT | 199 | 58 | 48 | 38 |
| | | AGATAATTCAAAGAGGAG | | | | |
| 28 | 4R | CGAGCGGCCGCCAGTGTGATGTGT | 199 | 62 | 52 | 38 |
| | | ACCTGATTCTCCATTTC | | | | |
| 29 | 5F | AGTAACGGCCGCCAGTGTGCTGTT | 193 | 62 | 52 | 38 |
| | | AGGTTGCAGTTTCATCAC | | | | |
| 30 | 5R | CGAGCGGCCGCCAGTGTGATGAAA | 193 | 64 | 54 | 38 |
| | | AGGTTAAGGGCTCTGAC | | | | |
| 31 | 6F | AGTAACGGCCGCCAGTGTGCTGGT | 221 | 62 | 52 | 39 |
| | | TTTCACTAATGAGCTTGC | | | | |
| 32 | 6R | CGAGCGGCCGCCAGTGTGATGCTA | 221 | 60 | 50 | 39 |
| | | TTCTGTTCTTATCCATG | | | | |
| 33 | 7F | AGTAACGGCCGCCAGTGTGCTGGT | 208 | 62 | 60 | 39 |
| | | AGAAGATGCAGAATTGAG | | | | |
| 34 | 7R | CGAGCGGCCGCCAGTGTGATGCAG | 208 | 62 | 60 | 39 |
| | | AGCCTGTATAACATTAG | | | | |
| 35 | 8F | AGTAACGGCCGCCAGTGTGCTGTT | 228 | 60 | 50 | 31 |
| | | CTTTTAGGAAAACACCAG | | | | |
| 36 | 8R | CGAGCGGCCGCCAGTGTGATGCTT | 228 | 62 | 52 | 31 |
| | | TCTTAAAGTGGCCTTTG | | | | |
| 37 | 9F | AGTAACGGCCGCCAGTGTGCTGCT | 208 | 64 | 54 | 36 |
| | | TTGTTCTGTTTGCAGGTG | | | | |
| 38 | 9R | CGAGCGGCCGCCAGTGTGATGCAA | 208 | 66 | 56 | 36 |
| | | CCTCCAATGACCCATTC | | | | |
| 39 | 10F | AGTAACGGCCGCCAGTGTGCTGTT | 196 | 62 | 52 | 35 |
| | | GTTTATCAAGGCTTGGAC | | | | |
| 40 | 10R | CGAGCGGCCGCCAGTGTGATGATT | 196 | 60 | 52 | 35 |
| | | TAACACCATTCTTCTGG | | | | |
| 41 | 11F | AGTAACGGCCGCCAGTGTGCTGCT | 195 | 62 | 52 | 29 |
| | | GTTATTTCGATTTGCAGC | | | | |
| 42 | 11R | CGAGCGGCCGCCAGTGTGATGGAC | 195 | 58 | 48 | 29 |
| | | ATTCAGAACATTATTAG | | | | |
| 43 | 12F | AGTAACGGCCGCCAGTGTGCTGAC | 203 | 62 | 52 | 41 |
| | | TCAATGATGTGTTAGCTC | | | | |
| 44 | 12R | CGAGCGGCCGCCAGTGTGATGTTC | 203 | 60 | 50 | 41 |
| | | ATCTTGAACTTCAACAC | | | | |
| 45 | 13F | AGTAACGGCCGCCAGTGTGCTGTC | 192 | 66 | 56 | 49 |
| | | GACAAACTGGGGTGATAG | | | | |
| 46 | 13R | CGAGCGGCCGCCAGTGTGATGTTT | 192 | 66 | 56 | 49 |
| | | CAGCCATGAACGTGGAG | | | | |
| 47 | 14F | AGTAACGGCCGCCAGTGTGCTGQG | 207 | 66 | 56 | 40 |
| | | AACTTCTACCTACGATGG | | | | |
| 48 | 14R | CGAGCGGCCGCCAGTGTGATGGTG | 207 | 68 | 58 | 40 |
| | | GTGAGTGCTGTGACATG | | | | |
| 49 | 15F | AGTAACGGCCGCCAGTGTGCTGTC | 211 | 62 | 52 | 40 |
| | | TTATAGGTGTCTGTGATC | | | | |
| 50 | 15R | CGAGCGGCCGCCAGTGTGATGCAC | 211 | 66 | 56 | 40 |
| | | TTCTTTGCTGCTGGTTC | | | | |
| 51 | 16F | AGTAACGGCCGCCAGTGTGCTGCC | 208 | 66 | 56 | 31 |
| | | AAGGTGAAACAAATGCCC | | | | |
| 52 | 16R | CGAGCGGCCGCCAGTGTCATGACC | 208 | 64 | 54 | 31 |
| | | TTCATTCCATTACTGGG | | | | |

TABLE 3

MSH2 exon probe sequences, gene ID, name
and size

SEQ

| | |
|---|---|
| ID | Probe |

| | | | | |
|---|---|---|---|---|
| No. | Name | Size | Exon | Sequence |

| | | | | |
|---|---|---|---|---|
| 53 | P1 | 198 | Promoter | gttttcaatc tgtcgcccac |
| | | | | gctggagtgc agtggcacaa |
| | | | | tttacggctg caccgcagcc |
| | | | | tcgacctccc gggctcaggt |
| | | | | gatcctttcg cctcagccct |
| | | | | gctaatatct gggatcacag |
| | | | | acgtgggttt taccatgttg |
| | | | | cccag |
| 54 | P2 | | Promoter | caatctcctg ggctcaagtg |
| | | | | atccgcccac ctcggcctcc |
| | | | | caaattgctg ggattacagg |
| | | | | cgtgagctac cgcgccctgc |
| | | | | cacaaacgca tatcttctaa |
| | | | | cgtaccattt catttacttg |
| | | | | ctatattcat tatctgaatt |
| | | | | ttctcatatt agaatgtaag |
| | | | | cagaataaag gcagt |
| 55 | S1 | 338 | 1 | gcgggaaaca gcttagtggg |
| | | | | tgtggggtcg cgcattttct |
| | | | | tcaaccagga ggtgaggagg |
| | | | | tttcGACATG GCGGTGCAGC |
| | | | | CGAAGGAGAC GCTGCAGTTG |
| | | | | GAGAGCCCGG CCGAGGTCGG |
| | | | | CTTCGTGCGC TTCTTTCAGG |
| | | | | GCATGCCGGA GAAGCCGACC |
| | | | | ACCACAGTGC GCCTTTTCGA |
| | | | | CCGGGGCGAC TTCTATACGG |
| | | | | CGCACGGCGA GGACGCGCTG |
| | | | | CTGGCCGCCC GGGAGGTGTT |
| | | | | CAAGACCCAG GGGGTGATCA |
| | | | | AGTACATGGG GCCGGCAGg |
| 56 | S2 | 220 | 2 | GGAGCAAAGA ATCTGCAGAG |
| | | | | TGTTGTGCTT AGTAAAATGA |
| | | | | ATTTTGAATC TTTTGTAAAA |
| | | | | GATCTTCTTC TGGTTCGTCA |
| | | | | GTATAGAGTT GAAGTTTATA |
| | | | | AGAATAGAGC TGGAAATAAG |
| | | | | GCATCAAGGA GAATGATTGG |
| | | | | TATTTGGCAT ATAAGg |
| 57 | S3 | 332 | 3 | CCTGGCAATC TCTCTCAGTT |
| | | | | TGAAGATATT CTCTTTGGTA |
| | | | | ACAATGATAT GTCAGCTTCC |
| | | | | ATTGGTGTTG TGGGTGTTAA |
| | | | | AATGTCCGCA GTTGATGGCC |
| | | | | AGAGACAGGT TGGAGTTGGG |
| | | | | TATGTGGATT CCATACAGAG |
| | | | | GAAACTAGGA CTGTGTGAAT |
| | | | | TCCCTGATAA TGATCAGTTC |
| | | | | TCCAATCTTG AGGCTCTCCT |
| | | | | CATCCAGATT GGACCAAAGG |
| | | | | AATGTGTTTT ACCCGGAGGA |
| | | | | GAGACTGCTG GAGACATGGG |
| | | | | GAAACTGAGA CAG |
| 58 | S4 | 220 | 4 | aaatagATAA TTCAAAGAGG |
| | | | | AGGAATTCTG ATCACAGAAA |
| | | | | GAAAAAAAGC TGACTTTTCC |
| | | | | ACAAAAGACA TTTATCAGGA |
| | | | | CCTCAACCGG TTGTTGAAAG |
| | | | | GCAAAAAGGG AGAGCAGATG |
| | | | | AATAGTGCTG TATTGCCAGA |
| | | | | AATGGAGAAT CAGgtacatg |
| | | | | g |
| 59 | S5 | 209 | 5 | GTTGCAGTTT CATCACTGTC |
| | | | | TGCGGTAATC AAGTTTTTAG |
| | | | | AACTCTTATC AGATGATTCC |
| | | | | AACTTTGGAC AGTTTGAACT |
| | | | | GACTACTTTT GACTTCAGCC |
| | | | | AGTATATGAA ATTGGATATT |
| | | | | GCAGCAGTCA GAGCCCTTAA |
| | | | | CCTTTTTCAG |
| 60 | S6 | 195 | 6 | gGGTTCTGTT GAAGATACCA |
| | | | | CTGGCTCTCA GTCTCTGGCT |
| | | | | GCCTTGCTGA ATAAGTGTAA |
| | | | | AACCCCTCAA GGACAAAGAC |
| | | | | TTGTTAACCA GTGGATTAAG |
| | | | | CAGCCTCTCA TGGATAAGAA |
| | | | | CAGAATAGAG GAGAGg |
| 61 | S7 | 263 | 7 | cagATTGAAT TTAGTGGAAG |
| | | | | CTTTTGTAGA AGATGCAGAA |
| | | | | TTGAGGCAGA CTTTACAAGA |
| | | | | AGATTTACTT CGTCGATTCC |
| | | | | CAGATCTTAA CCGACTTGCC |
| | | | | AAGAAGTTTC AAAGACAAGC |
| | | | | AGCAAACTTA CAAGATTGTT |
| | | | | ACCGACTCTA TCAGGGTATA |
| | | | | AATCAACTAC CTAATGTTATA |
| | | | | CAGGCTCTGG AAAAACATGA |
| | | | | Agg |
| 62 | S8 | 176 | 8 | gGAAAACACC AGAAATTATT |
| | | | | GTTGGCAGTT TTTGTGACTC |
| | | | | CTCTTACTGA TCTTCGTTCT |
| | | | | GACTTCTCCA AGTTTCAGGA |
| | | | | AATGATAGAA ACAACTTTAG |
| | | | | ATATGGATCA Ggtatgc |
| 63 | S9 | 188 | 9 | gcagGTGGAA AACCATGAAT |
| | | | | TCCTTGTAAA ACCTTCATTT |
| | | | | GATCCTAATC TCAGTGAATT |
| | | | | AAGAGAAATA ATGAATGACT |
| | | | | TGGAAAAGAA GATGCAGTCA |
| | | | | ACATTAATAA GTGCAGCCAG |
| | | | | AGATCTTGg |
| 64 | S10 | 249 | 10 | gtttatcaag GGCTTGGACC |
| | | | | CTGGCAAACA GATTAAACTG |
| | | | | GATTCCAGTG CACAGTTTGG |
| | | | | ATATTACTTT CGTGTAACCT |
| | | | | GTAAGGAAGA AAAAGTCCTT |
| | | | | CGTAACAATA AAAACTTTAG |
| | | | | TACTGTAGAT ATCCAGAAGA |
| | | | | ATGGTGTTAA ATTTACCAAC |
| | | | | Aggtttgcaa gtcgttatta |
| | | | | tatttttaac c |
| 65 | S11 | 159 | 11 | GAAGCCCAGG ATGCCATTGT |
| | | | | TAAAGAAATT GTCAATATTT |
| | | | | CTTCAGgtaa acttaataga |
| | | | | actaataatg ttctgaatgt |
| | | | | cacctggctt ttggtaacag |
| 66 | S12 | 307 | 12 | GGCTATGTAG AACCAATGCA |
| | | | | GACACTCAAT GATGTGTTAG |
| | | | | CTCAGCTAGA TGCTGTTGTC |
| | | | | AGCTTTGCTC ACGTGTCAAA |
| | | | | TGGAGCACCT GTTCCATATG |
| | | | | TACGACCAGC CATTTTGGAG |
| | | | | AAAGGACAAG GAAGAATTAT |
| | | | | ATTAAAAGCA TCCAGGCATG |
| | | | | CTTGTGTTGA AGTTCAAGAT |
| | | | | GAAATTGCAT TTATTCCTAA |
| | | | | TGACGTATAC TTTGAAAAAG |
| | | | | ATAAACAGAT GTTCCACATC |
| | | | | ATTACTGg |
| 67 | S13 | 266 | 13 | gGCCCCAATA TGGGAGGTAA |
| | | | | ATCAACATAT ATTCGACAAA |
| | | | | CTGGGGTGAT AGTACTCATG |
| | | | | GCCCAAATTG GGTGTTTTGT |
| | | | | GCCATGTGAG TCAGCAGAAG |
| | | | | TGTCCATTGT GGACTGCATC |
| | | | | TTAGCCCGAG TAGGGGCTGG |
| | | | | TGACAGTCAA TTGAAAGGAG |
| | | | | TCTCCACGTT CATGGCTGAA |
| | | | | ATGTTGGAAA CTGCTTCTAT |
| | | | | CCTCAGg |
| 68 | S14 | 315 | 14 | cagGTCTGCA ACCAAAGATT |
| | | | | CATTAATAAT CATAGATGAA |
| | | | | TTGGGAAGAG GAACTTCTAC |
| | | | | CTACGATGGA TTTGGGTTAG |
| | | | | CATGGGCTAT ATCAGAATAC |
| | | | | ATTGCAACAA AGATTGGTGC |
| | | | | TTTTTGCATG TTTGCAACCC |
| | | | | ATTTTCATGA ACTTACTGCC |
| | | | | TTGGCCAATC AGATACCAAC |
| | | | | TGTTAATAAT CTACATGTCA |
| | | | | CAGCACTCAC CACTGAAGAG |
| | | | | ACCTTAACTA TGCTTTATCA |
| | | | | GGTGAAGAAA Ggtatg |
| 69 | S15 | 237 | 15 | gGTGTCTGTG ATCAAAGTTT |
| | | | | TGGGATTCAT GTTGCAGAGC |
| | | | | TTGCTAATTT CCCTAAGCAT |
| | | | | GTAATAGAGT GTGCTAAACA |
| | | | | GAAAGCCCTG GAACTTGAGG |
| | | | | AGTTTCAGTA TATTGGAGAA |
| | | | | TCGCAAGGAT ATGATATCAT |
| | | | | GGAACCAGCA GCAAAGAAGT |
| | | | | GCTATCTGGA AAGAGAGg |
| 70 | S16 | 153 | 16 | ctcatgggac attcacatgt |
| | | | | gtttcagcAA GGTGAAAAAA |
| | | | | TTATTCAGGA GTTCCTGTCC |
| | | | | AAGGTGAAAC AAATGCCCTT |
| | | | | TACTGAAATG TCAG |

TABLE 4

MLH1 exon probe sequences, gene ID, name
and size

SEQ

| | |
|---|---|
| ID | Probe |

| | | | | |
|---|---|---|---|---|
| No. | Name | Size | Exon | Sequence |

| | | | | |
|---|---|---|---|---|
| 71 | L1 | 243 | 1 | CTTCCGTTGA GCATCTAGAC |
| | | | | GTTTCCTTGG CTCTTCTGGC |
| | | | | GCCAAAATGT CGTTCGTGGC |
| | | | | AGGGGTTATT CGGCGGCTGG |
| | | | | ACGAGACAGT GGTGAACCGC |
| | | | | ATCGCGGCGG GGGAAGTTAT |
| | | | | CCAGCGGCCA GCTAATGCTA |
| | | | | TCAAAGAGAT GATTGAGAAC |
| | | | | Tggtacggag ggagtcgagc |
| | | | | cggg |
| 72 | L2 | 276 | 2 | ctcatattaa aatatgtaca |
| | | | | ttagagtagt tgcagactga |
| | | | | taaattattt tctgtttgat |
| | | | | ttgccagTTT AGATGCAAAA |
| | | | | TCCACAAGTA TTCAAGTGAT |
| | | | | TGTTAAAGAG GGAGGCCTGA |
| | | | | AGTTGATTCA GATCCAAGAC |
| | | | | AATGGCACCG GGATCAGGgt |
| | | | | aagtaaaacc tcaaagtagc |
| | | | | aggatgtttg tgcgcttcat |
| | | | | ggaagagtca ggacctttct |
| | | | | c |
| 73 | L3 | 224 | 3 | gagatttgga aaaatgagta |
| | | | | acatgattat ttactcatct |
| | | | | ttttggtatc taacagAAAG |
| | | | | AAGATCTGGA TATTGTATGT |
| | | | | GAAAGGTTCA CTACTAGTAA |
| | | | | ACTGCAGTCC TTTGAGGATT |
| | | | | TAGCCAGTAT TTCTACCTAT |
| | | | | GGCTTTCGAG GTGAGgtaag |
| | | | | ctgag |
| 74 | L4 | 171 | 4 | cttttcttcc ttagGCTTTG |
| | | | | GCCAGCATAA GCCATGTGGC |
| | | | | TCATGTTACT ATTACAACGA |
| | | | | AAACAGCTGA TGGAAAGTGT |
| | | | | GCATACAGgt atagtgctga |
| | | | | cttcttttac tc |
| 75 | L5 | 288 | 5 | ttgatatgat tttctctttt |
| | | | | ccccttggga ttagtatcta |
| | | | | tctctctact ggatattaat |
| | | | | ttgttatatt ttctcattag |
| | | | | AGCAAGTTAC TCAGATGGAA |
| | | | | AACTGAAAGC CCCTCCTAAA |
| | | | | CCATGTGCTG GCAATCAAGG |
| | | | | GACCCAGATC A |
| 76 | L6 | 257 | 6 | gggttttatt ttcaagtact |
| | | | | tctatgaatt tacaagaaaa |
| | | | | atcaatcttc tgttcagGTG |
| | | | | GAGGACCTTT TTTACAACAT |
| | | | | AGCCACGAGG AGAAAAGCTT |
| | | | | TAAAAAATCC AAGTGAAGAA |
| | | | | TATGGGAAAA TTTTGGAAGT |
| | | | | TGTTGGCAGg tacagtccaa |
| | | | | aatctgggag tgggtctctg |
| | | | | agatttgtca tcaaa |
| 77 | L7 | 167 | 7 | ggctctgaca tctagtgtgt |
| | | | | gtttttggca actcttttct |
| | | | | tactcttttg tttttctttt |
| | | | | ccagGTATTC AGTACACAAT |
| | | | | GCAGGCATTA GTTTCTCAGT |
| | | | | TAAAAAAg |
| 78 | L8 | 149 | 8 | CAAGGAGAGA CAGTAGCTGA |
| | | | | TGTTAGGACA CTACCCAATG |
| | | | | CCTCAACCGT GGACAATATT |
| | | | | CGCTCCGTCT TTGGAAATGC |
| | | | | TGTTAGTCGg |
| 79 | L9 | 184 | 9 | GAGAACTGAT AGAAATTGGA |
| | | | | TGTGAGGATA AAACCCTAGC |
| | | | | CTTCAAAATG AATGGTTACA |
| | | | | TATCCAATGC AAACTACTCA |
| | | | | GTGAAGAAGT GCATCTTCTT |
| | | | | ACTCTTCATC AACCgtaagt |
| | | | | taaaa |
| 80 | L10 | 327 | 10 | ttattgttta gATCGTCTGG |
| | | | | TAGAATCAAC TTCCTTGAGA |
| | | | | AAAGCCATAG AAACAGTGTA |
| | | | | TGCAGCCTAT TTGCCCAAAA |
| | | | | ACACACACCC ATTCCTGTAC |
| | | | | CTCAGgtaat gtagcaccaa |
| | | | | actcctcaac caagactcac |
| | | | | aaggaacaga tgttctatca |
| | | | | ggctctcctc tttgaaagag |
| | | | | atgagcatgc taatagtaca |
| | | | | atcagagtga atcccataca |
| | | | | ccactggcaa aaggatgttc |
| | | | | tgtcccttct tacaggtaca |
| | | | | aggcacag |
| 81 | L11 | 274 | 11 | cctgacagTT TAGAAATCAG |
| | | | | TCCCCAGAAT GTGGATGTTA |
| | | | | ATGTGCACCC CACAAAGCAT |
| | | | | GAAGTTCACT TCCTGCACGA |
| | | | | GGAGAGCATC CTGGAGCGGG |
| | | | | TGCAGCAGCA CATCGAGAGC |
| | | | | AAGCTCCTGG GCTCCAATTC |
| | | | | CTCCAGGATG TACTTCACCC |
| | | | | Aggtcagggc gcttctcatc |
| | | | | cagctacttc tctctggggc |
| | | | | ctttgaaatg tgcccgg |
| 82 | L12 | 359 | 12 | CCTCTGGGGA GATGGTTAAAT |
| | | | | CCACAACAAGT CTGACCTCGT |
| | | | | CTTCTACTTC TGGAAGTAGT |
| | | | | GATAAGGTCT ATGCCCACCA |
| | | | | GATGGTTCGT ACAGATTCCC |
| | | | | GGGAACAGAA GCTTGATGCA |
| | | | | TTTCTGCAGC CTCTGAGCAA |
| | | | | ACCCCTGTCC AGTCAGCCCC |
| | | | | AGGCCATTGT CACAGAGGAT |
| | | | | AAGACAGATA TTTCTAGTGG |
| | | | | CAGGGCTAGG CAGCAAGATG |
| | | | | AGGAGATGCT TGAACTCCCA |
| | | | | GCCCCTGCTG AAGTGGCTGC |
| | | | | CAAAAATCAG AGCTTGGAGG |
| | | | | GGGATACAAC AAAGGGGA |
| 83 | L13 | 257 | 13 | ccttttcttc attgcagAAA |
| | | | | GAGACATCGG GAAGATTCTG |
| | | | | ATGTGGAAAT GGTGGAAGAT |
| | | | | GATTCCCGAA AGGAAATGAC |
| | | | | TGCAGCTTGT ACCCCCCGGA |
| | | | | GAAGGATCAT TAACCTCACT |
| | | | | AGTGTTTTGA GTCTCCAGGA |
| | | | | AGAAATTAAT GAGCAGGGAC |
| | | | | ATGAGGgtac gtaaacgctg |
| | | | | tggcctgcct gggatgcata |
| | | | | ggg |
| 84 | L14 | 205 | 14 | gcagTTCTCC GGGAGATGTT |
| | | | | GCATAACCAC TCCTTCGTGG |
| | | | | GCTGTGTGAA TCCTCAGTGG |
| | | | | GCCTTGGCAC AGCATCAAAC |
| | | | | CAAGTTATAC CTTCTCAACA |
| | | | | CCACCAAGCT TAGataaatc |
| | | | | agctgagtgt gtgtaacaag |
| | | | | cagagct |
| 85 | L15 | 199 | 15 | cagTGAAGAA CTGTTCTACC |
| | | | | AGATACTCAT TTATGATTTT |
| | | | | GCCAATTTTG GTGTTCTCAG |
| | | | | GTTATCGgta agtttagatc |
| | | | | cttttcactt ctgacatttc |
| | | | | aactgaccgc cccgcaaaca |
| | | | | gtagctctcc actaaata |
| 86 | L16 | 303 | 16 | cctagGAGCC AGCACCGCTC |
| | | | | TTTGACCTTG CCATGCTTGC |
| | | | | CTTAGATAGT CCAGAGAGTG |
| | | | | GCTGGACAGA GGAAGATGGT |
| | | | | CCCAAAGAAG GACTTGCTGA |
| | | | | ATACATTGTT GAGTTTCTGA |
| | | | | AGAAGAAGGC TGAGATGCTT |
| | | | | GCAGACTATT TCTCTTTGGA |
| | | | | AATTGATGAG gtgtgacagc |
| | | | | cattcttata cttctgttgt |
| | | | | attctccaaa taaaatttcc |
| | | | | agccgggtgc attggc |
| 87 | L17 | 311 | 17 | gttcccttgt cctttttcct |
| | | | | gcaagcagGA AGGGAACCTG |
| | | | | ATTGGATTAC CCCTTCTGAT |
| | | | | TGACAACTAT GTGCCCCCTT |
| | | | | TGGAGGGACT GCCTATCTTC |
| | | | | ATTCTTCGAC TAGCCACTGA |
| | | | | Ggtcagtgat caagcagata |
| | | | | ctaagcattt cggtacatgc |
| | | | | atgtgtgctg gagggaa |
| 88 | L18 | 229 | 18 | gaggtattga atttctttgg |
| | | | | accagGTGAA TTGGGACGAA |
| | | | | GAAAAGGAAT GTTTTGAAAG |
| | | | | CCTCAGTAAA GAATGCGCTA |
| | | | | TGTTCTATTC CATCCGGAAG |
| | | | | CAGTACATAT CTGAGGAGTC |
| | | | | GACCCTCTAG GCCAGCAGgt |
| | | | | acagtgggta tgacactggc |
| | | | | accccaggac |
| 89 | L19 | 322 | 19 | ccagAGTGA AGTGCCTGG |
| | | | | CTCCATTCC AAACTCCTG |
| | | | | GAAGTGGAC TGTGGAACA |
| | | | | CATTGTCTA TAAAGCCTT |
| | | | | GCGCTCACA CATTCTGCC |
| | | | | TCCTAAACA TTTCACAGA |
| | | | | AGATGGAAA TATCCTGCA |
| | | | | GCTTGCTAA CCTGCCTGA |
| | | | | TCTATACAA AGTCTTTGA |
| | | | | GAGGTGTTA AATATGGTT |
| | | | | ATTTATGCA CTGTGGGAT |
| | | | | GTGTTCTTC TTTCTCTGT |
| | | | | ATTCCGATA CAAAGTGTT |
| | | | | GTATCAAAG TGTGATATA |
| | | | | CAAAGTGTA CC |

TABLE 5

60-mer Oligonucleotides of control, MSH2
and MLH1 for production of HNPCC
microarrays

SEQ

| | | |
|---|---|---|
| ID | Oligo | GC- |

| | | | | | |
|---|---|---|---|---|---|
| No. | Gene ID | Name | content | Exon | Sequence |

| | | | | | |
|---|---|---|---|---|---|
| 90 | sub- | 8D2-1 | 58% | Control | TGGCTTTCTAC |
| | telomeric | | | | TCCCGCAACCA |
| | | | | | GGAGCCTGACT |
| | | | | | AACAAGGGGAT |
| | | | | | GGCAGGCGACC |
| | | | | | ACATC |
| 91 | sub- | 8B4-2 | 51% | Control | GAAATGATACC |
| | telomeric | | | | CATGGGAACAG |
| | | | | | AGAAACCTGCG |
| | | | | | TGTGAGGTGTC |
| | | | | | AGCATGAGGAG |
| | | | | | ACCAG |
| 92 | sub- | TBX5B1-3 | 56% | Control | CCAGCAGGTAA |
| | telomeric | | | | GGAGACCTCGC |
| | | | | | GCTTCGGGTCC |
| | | | | | CTTTGCAGAGA |
| | | | | | TCAAAGTCAGA |
| | | | | | GTCTG |
| 93 | sub- | 10B4D-4 | 61% | Control | CTGGTGAGGGT |
| | telomeric | | | | CACTTTCAGGC |
| | | | | | TGCTTAGACCT |
| | | | | | GCAGCCTCCCC |
| | | | | | AGGTGGTGTGC |
| | | | | | TCAGG |
| 94 | TBX gene | TBX5Da-5 | 55% | Control | CTGGTGAGGGT |
| | | | | | CACTTTCAGGC |
| | | | | | TGCTTAGACCT |
| | | | | | GCAGCCTCCCC |
| | | | | | AGGTGGTGTGC |
| | | | | | TCAGG |
| 95 | sub-telomeric | 5C3-6 | 55% | Control | GACCTTAAGAG |
| | | | | | CAGGGAGGTCA |
| | | | | | GAAGCCCTGTG |
| | | | | | GGCTGAGTAAT |
| | | | | | CCTCTGAAGCA |
| | | | | | CTTGC |
| 96 | sub- | 4A4-7 | 55% | Control | CACAGGCCAGG |
| | telomeric | | | | AGACCAACCTC |
| | | | | | TAACCCTGATC |
| | | | | | TGACACAGGTC |
| | | | | | TAAGGGGAAGG |
| | | | | | TCATG |
| 97 | Y-linked | A2Y-8 | 48% | Control | GAAAACAGTAA |
| | | | | | AGGCAACGTCC |
| | | | | | AGGATAGAGTG |
| | | | | | AAGCGACCCAT |
| | | | | | GAACGCATTCA |
| | | | | | TCGTG |
| 98 | sub- | 11G9-9 | 56% | Control | GTAGCCGTATG |
| | telomeric | | | | TGAACCATGGG |
| | | | | | GCAAGGTGGTC |
| | | | | | AGCGGGGGTCA |
| | | | | | GAGGTATTGTA |
| | | | | | CAAGG |
| 99 | TBX gene | TBX5A2-10 | 53% | Control | CACTCCTGGCT |
| | | | | | TTTGCGCTCCA |
| | | | | | AAGAAGAGGTG |
| | | | | | GGATAGTTGGA |
| | | | | | GGTGAGTTTCA |
| | | | | | CCCTG |
| 100 | X-linked | 2D2-11 | 56% | Control | CTCAGCACTCC |
| | | | | | TGGGATTTGGA |
| | | | | | ACCTCGTTCCT |
| | | | | | CTCTGCAAAGC |
| | | | | | CTCCTAGCCCG |
| | | | | | GTTCT |
| 101 | Non-human | D11-12 | 46% | Control | CTAATCAAGTT |
| | | | | | TTTTGGGGTCG |
| | | | | | AGGTGCCGTAA |
| | | | | | AGCACTAAATC |
| | | | | | GGAACCCTAAA |
| | | | | | GGGAG |
| 102 | MSH2 | MSH2-p1 | 58% | Promoter | GTTTTCAATCT |
| | | | | | GTCGCCCACGC |
| | | | | | TGGAGTGCAGT |
| | | | | | GGCACAATTTA |
| | | | | | CGGCTG CACC |
| | | | | | GCAGCC |
| 103 | MSH2 | MSH2-p2 | 51% | Promoter | CAAATTGCTGG |
| | | | | | GATTACAGGCG |
| | | | | | TGAGCTACCGC |
| | | | | | GCCCTGCCACA |
| | | | | | AACGCATATCT |
| | | | | | TCTAA |
| 104 | MSH2 | MSH2-1 | 60% | 1 | CTTCGTGCGCT |
| | | | | | TCTTTCAGGGC |
| | | | | | ATGCCGGAGAA |
| | | | | | GCCGACCACCA |
| | | | | | CAGTGCGCCTT |
| | | | | | TTCGA |
| 105 | MSH2 | MSH2-2 | 31% | 2 | CTGCAGAGTGT |
| | | | | | TGTGCTTAGTA |
| | | | | | AAATGAATTTT |
| | | | | | GAATCTTTTGT |
| | | | | | AAAAGATCTTC |
| | | | | | TTCTG |
| 106 | MSH2 | MSH2-3 | 46% | 3 | GACAGGTTGGA |
| | | | | | GTTGGGTATGT |
| | | | | | GGATTCCATAC |
| | | | | | AGAGGAAACTA |
| | | | | | GGACTGTGTGA |
| | | | | | ATTCC |
| 107 | MSH2 | MSH2-4 | 48% | 4 | GGACCTCAACC |
| | | | | | GGTTGTTGAAA |
| | | | | | GGCAAAAAGGG |
| | | | | | AGAGCAGATGA |
| | | | | | ATAGTGCTGTA |
| | | | | | TTGCC |
| 108 | MSH2 | MSH2-5 | 41% | 5 | GAACTGACTAC |
| | | | | | TTTTGACTTCA |
| | | | | | GCCAGTATATG |
| | | | | | AAATTGGATAT |
| | | | | | TGCAGCAGTCA |
| | | | | | GAGCC |
| 109 | MSH2 | MSH2-6 | 48% | 6 | CTGTTGAAGAT |
| | | | | | ACCACTGGCTC |
| | | | | | TCAGTCTCTGG |
| | | | | | CTGCCTTGCTG |
| | | | | | AATAAGTGTAA |
| | | | | | AACCC |
| 110 | MSH2 | MSH2-7 | 45% | 7 | CTTCGTCGATT |
| | | | | | CCCAGATCTTA |
| | | | | | ACCGACTTGCC |
| | | | | | AAGAAGTTTCA |
| | | | | | AAGACAAGCAG |
| | | | | | CAAAC |
| 111 | MSH2 | MSH2-8 | 41% | 8 | GCAGTTTTTGT |
| | | | | | GACTCCTCTTA |
| | | | | | CTGATCTTCGT |
| | | | | | TCTGACTTCTC |
| | | | | | CAAGTTTCAGG |
| | | | | | AAATG |
| 112 | MSH2 | MSH2-9 | 35% | 9 | GGTGGAAAACC |
| | | | | | ATGAATTCCTT |
| | | | | | GTAAAACCTTC |
| | | | | | ATTTGATCCTA |
| | | | | | ATCTCAGTGAA |
| | | | | | TTAAG |
| 113 | MSH2 | MSH2-10 | 43% | 10 | GCTTGGACCCT |
| | | | | | GGCAAACAGAT |
| | | | | | TAAACTGGATT |
| | | | | | CCAGTGCACAG |
| | | | | | TTTGGATATTA |
| | | | | | CTTTC |
| 114 | MSH2 | MSH2-11 | 33% | 11 | GACTTCTTTAA |
| | | | | | ATGAAGAGTAT |
| | | | | | ACCAAAAATAA |
| | | | | | AACAGAATATG |
| | | | | | AAGAAGCCCAG |
| | | | | | GATGC |
| 115 | MSH2 | MSH2-12 | 48% | 12 | CTCAGCTAGAT |
| | | | | | GCTGTTGTCAG |
| | | | | | CTTTGCTCACG |
| | | | | | TGTCAAATGGA |
| | | | | | GCACCTGTTCC |
| | | | | | ATATG |
| 116 | MSH2 | MSH2-13 | 51% | 13 | GGTGTTTTGTG |
| | | | | | CCATGTGAGTC |
| | | | | | AGCAGAAGTGT |
| | | | | | CCATTGTGGAC |
| | | | | | TGCATCTTAGC |
| | | | | | CCGAG |
| 117 | MSH2 | MSH2-14 | 41% | 14 | GAACTTACTGC |
| | | | | | CTTGGCCAATC |
| | | | | | AGATACCAACT |
| | | | | | GTTAATAATCT |
| | | | | | ACATGTCACAG |
| | | | | | CACTC |
| 118 | MSH2 | MSH2-15 | 41% | 15 | GGGATTCATGT |
| | | | | | TGCAGAGCTTG |
| | | | | | CTAATTTCCCT |
| | | | | | AAGCATGTAAT |
| | | | | | AGAGTGTGCTA |
| | | | | | AACAG |
| 119 | MSH2 | MSH2-16 | 33% | 16 | GCCCTTTACTG |
| | | | | | AAATGTCAGAA |
| | | | | | GAAAACATCAC |
| | | | | | AATAAAGTTAA |
| | | | | | AACAGCTAAAA |
| | | | | | GCTGA |
| 120 | MLH1 | MLH1-1 | 56% | 1 | CAAAATGTCGT |
| | | | | | TCGTGGCAGGG |
| | | | | | GTTATTCGGCG |
| | | | | | GCTGGACGAGA |
| | | | | | CAGTGGTGAAC |
| | | | | | CGCAT |
| 121 | MLH1 | MLH1-2 | 41% | 2 | CAAGTGATTGT |
| | | | | | TAAAGAGGGAG |
| | | | | | GCCTGAAGTTG |
| | | | | | ATTCAGATCCA |
| | | | | | AGACAATGGCA |
| | | | | | CCGGG |
| 122 | MLH1 | MLH1-3 | 41% | 3 | GGTTCACTACT |
| | | | | | AGTAAACTGCA |
| | | | | | GTCCTTTGAGG |
| | | | | | ATTTAGCCAGT |
| | | | | | ATTTCTACCTA |
| | | | | | TGGCT |
| 123 | MLH1 | MLH1-4 | 45% | 4 | GGCCAGCATAA |
| | | | | | GCCATGTGGCT |
| | | | | | CATGTTACTAT |
| | | | | | TACAACGAAAA |
| | | | | | CAGCTGATGGA |
| | | | | | AAGTG |
| 124 | MLH1 | MLH1-5 | 48% | 5 | GCAAGTTACTC |
| | | | | | AGATGGAAAAC |
| | | | | | TGAAAGCCCCT |
| | | | | | CCTAAACCATG |
| | | | | | TGCTGGCAATC |
| | | | | | AAGGG |
| 125 | MLH1 | MLH1-6 | 38% | 6 | GGAGGACCTTT |
| | | | | | TTTACAACATA |
| | | | | | GCCACGAGGAG |
| | | | | | AAAAGCTTTAA |
| | | | | | AAAATCCAAGT |
| | | | | | GAAGA |
| 126 | MLH1 | MLH1-7 | 35% | 7 | CTCTTTTGTTT |
| | | | | | TTCTTTTCCAG |
| | | | | | GTATTCAGTAC |
| | | | | | ACAATGCAGGC |
| | | | | | ATTAGTTTCTC |
| | | | | | AGTTA |
| 127 | MLH1 | MLH1-8 | 50% | 8 | GACAGTAGCTG |
| | | | | | ATGTTAGGACA |
| | | | | | CTACCCAATGC |
| | | | | | CTCAACCGTGG |
| | | | | | ACAATATTCGC |
| | | | | | TCCGT |
| 128 | MLH1 | MLH1-9 | 40% | 9 | CCCTAGCCTTC |
| | | | | | AAAATGAATGG |
| | | | | | TTACATATCCA |
| | | | | | ATGCAAACTAC |
| | | | | | TCAGTGAAGAA |
| | | | | | GTGCA |
| 129 | MLH1 | MLH1-10 | 46% | 10 | GCCATAGAAAC |
| | | | | | AGTGTATGCAG |
| | | | | | CCTATTTGCCC |
| | | | | | AAAAACACACA |
| | | | | | CCCATTCCTGT |
| | | | | | ACCTC |
| 130 | MLH1 | MLH1-11 | 56% | 11 | CAAAGCATGAA |
| | | | | | GTTCACTTCCT |
| | | | | | GCACGAGGAGA |
| | | | | | GCATCCTGGAG |
| | | | | | CGGGTGCAGCA |
| | | | | | GCACA |
| 131 | MLH1 | MLH1-12 | 51% | 12 | GGTTCGTACAG |
| | | | | | ATTCCCGGGAA |
| | | | | | CAGAAGCTTGA |
| | | | | | TGCATTTCTGC |
| | | | | | AGCCTCTGAGC |
| | | | | | AAACC |
| 132 | MLH1 | MLH1-13 | 45% | 13 | CATCGGGAAGA |
| | | | | | TTCTGATGTGG |
| | | | | | AAATGGTGGAA |
| | | | | | GATGATTCCCG |
| | | | | | AAAGGAAATGA |
| | | | | | CTGCA |
| 133 | MLH1 | MLH1-14 | 55% | 14 | GCATAACCACT |
| | | | | | CCTTCGTGGGC |
| | | | | | TGTGTGAATCC |
| | | | | | TCAGTGGGCCT |
| | | | | | TGGCACAGCAT |
| | | | | | CAAAC |
| 134 | MLH1 | MLH1-15 | 36% | 15 | GAACTGTTCTA |
| | | | | | CCAGATACTCA |
| | | | | | TTTATGATTTT |
| | | | | | GCCAATTTTGG |
| | | | | | TGTTCTCAGGT |
| | | | | | TATCG |
| 135 | MLH1 | MLH1-16 | 55% | 16 | GACCTTGCCAT |
| | | | | | GCTTGCCTTAG |
| | | | | | ATAGTCCAGAG |
| | | | | | AGTGGCTGGAC |
| | | | | | AGAGGAAGATG |
| | | | | | GTCCC |
| 136 | MLH1 | MLH1-17 | 51% | 17 | GGGAACCTGAT |
| | | | | | TGGATTACCCC |
| | | | | | TTCTGATTGAC |
| | | | | | AACTATGTGCC |
| | | | | | CCCTTTGGAGG |
| | | | | | GACTG |
| 137 | MLH1 | MLH1-18 | 45% | 18 | CCTCAGTAAAG |
| | | | | | AATGCGCTATG |
| | | | | | TTCTATTCCAT |
| | | | | | CCGGAAGCAGT |
| | | | | | ACATATCTGAG |
| | | | | | GAGTC |
| 138 | MLH1 | MLH1-19 | 50% | 19 | CTCCTGGAAGT |
| | | | | | GGACTGTGGAA |
| | | | | | CACATTGTCTA |
| | | | | | TAAAGCCTTGC |
| | | | | | GCTCACACATT |
| | | | | | CTGCC |

1-35. (canceled)

36. A method for hybridization of probes onto immobilized genomic DNA comprising the steps of:
(a) providing a sample containing or suspected of having genomic content, wherein said genomic content is undigested or intact chromosomal DNA or RNA;
(b) denaturing said intact genomic content;
(c) immobilizing said denatured intact genomic content within matrix; said matrix comprising pore sizes within a range of 0.6 μm to 2 μm including the outer limits;
(d) providing a set of probes and passing said probes through said matrix under conditions favoring hybridization of the probes to its complementary sequence in said intact genomic content; and
(e) washing off non-hybridized probes through said matrix, leaving formed hybridized intact genomic content/probe complexes for further analysis.

37. The method according to claim 36, wherein said denatured intact genomic DNA is permeated within said matrix.

38. The method according to claim 36, wherein said probes are passed through said matrix by at least one cycle of alternating downwards and upwards flow.

39. The method according to claim 36, wherein said washing step is carried out by passing through said matrix a wash fluid by at least one cycle of downwards flow.

40. The method according to claim 36, wherein said matrix is a membrane.

41. The method according to claim 40, wherein said membrane comprises a 3D network structure.

42. The method according to claim 41, wherein said network structure is a flow-through structure.

43. The method according to claim 41, wherein said network structure is a fibre network structure.

44. The method according to claim 43, wherein said fibre is of vegetable origin.

45. The method according to claim 44, wherein said fibre is cellulose.

46. The method according to claim 36, wherein the matrix allows for a flow rate comprised between 50 mm/30 min and 250 mm/30 min including the outer limits.

47. The method according to claim 36, wherein said matrix is activated with an affinity conjugate.

48. The method according to claim 47, wherein said affinity conjugate is chosen from the group comprising poly-L-lysine, poly-D-lysine, 3-aminopropyl-triethoxysilane, poly-arginine, polyethyleneimine, polyvinylamine, polyallylamine, tetraethylenepentamine, ethylenediamine, diethylenetriamine, triethylenetetramine, pentaethylenehexamine and hexamethylenediamine.

49. The method according to claim 48, wherein said affinity conjugate is poly-L-lysine.

50. The method according to claim 36, wherein said probes are flanked by primer binding sequences.

51. A method for target nucleic acid detection and quantification in an intact genomic DNA sample comprising the steps of:
(a) providing intact genomic DNA and denaturing said intact genomic DNA;
(b) performing a hybridization according to the method of claim 36;
(c) recovering hybridized probes; and essentially simultaneously amplifying any recovered probe using a single primer pair, each member of said primer pair binding to each recovered probe onto the respective flanking primer binding sequences of said probe; and
(d) qualitatively and quantitatively analyzing the recovered amplified probes of step (c).

52. The method according to claim 51, wherein the analysis of step (d) is by microarray analysis.

53. The method according to claim 51, wherein each probe is flanked 5′ and 3′ by primer binding regions with said 5′ and 3′ flanking primer binding sequences being the same or substantially the same for each probe.

54. The method according to claim 51, wherein said amplification of step (c) is a quantitative amplification.

55. The method according to claim 54, wherein said amplification is by means of polymerase chain reaction.

56. The method according to claim 51, wherein the amplified probes are provided with a label.

57. The method according to claim 56, wherein said label is a fluorescent label.

58. A device for flow-through hybridization of probes onto immobilized intact genomic DNA comprising a well holder, said well holder comprising one or more round wells with a fixed diameter, said wells exposing a fibre network matrix, said matrix comprising pore sizes within a range of 0.6 μm to 2 μm including the outer limits; wherein said matrix permits immobilization of intact genomic DNA and which allows hybridization of said immobilized intact genomic material with probes by flow-through hybridization.

59. The device according to claim 58, wherein said matrix permits permeation of intact genomic DNA.

60. An apparatus for flow-through hybridization of probes onto immobilized genomic DNA comprising:
(a) a device according to claim 58;
(b) means for addition of a controlled amount of fluid to at least one of the wells of the device as described in (a);
(c) means for applying and/or maintaining a controlled pressure difference over the matrix in each of the wells.

61. A kit for flow-through hybridization of probes onto immobilized intact genomic DNA comprising:
(a) a device according to claim 58; and
(b) instructions.

62. A kit according to claim 61, additionally comprising:
(a) a set of probes, wherein each probe is flanked 5′ end 3′ by primer binding regions with said 5′ and 3′ flanking primer binding sequences being the same or substantially the same for each probe;
(b) a single primer pair, each member of said pair being complementary to a primer binding region;
(c) optionally amplification components allowing the amplification of any recovered hybridized probe; and
(d) optionally a microarray, said microarray allowing analysis of the hybridization results.
